(19) United States
(12) Patent Application Publication
(10) Pub. No.: US 2007/0105837 A1
(43) Pub. Date: May 10, 2007

(54) Novel derivatives of 4a,5,9,10,11,12-hexahydrobenzofuro[3a,3,2][2]-benzazepine, method for the production thereof and use thereof in the production of medicaments

(76) Inventors: Laszlo Czollner, Ebenfurth (AT); Beate Kalz, Steinbrunn (AT); Stefan Welzig, Wien (AT); J. Werner Frantsits, Wien (AT); Ulrich Jordis, Wien (AT); Johannes Frohlich, Dornbach/Wienerwald (AT)

Correspondence Address: POPOVICH, WILES & O'CONNELL, PA;650 THIRD AVENUE SOUTH, SUITE 600, MINNEAPOLIS, MN, 55402, US

(21) Appl. No.: 10/573,517

(22) PCT Filed: Sep. 9, 2004
(86) PCT No.: PCT/AT04/00309

§ 371 Date: Jun. 5, 2006

(30) Foreign Application Priority Data
Sep. 29, 2003 (AT) A 1538/2003
Jul. 12, 2004 (AT) A 1174/2004

(51) Int. Cl. A61K 31/55; C07D 491/02

(52) U.S. Cl. 514/215; 540/576

(57) ABSTRACT

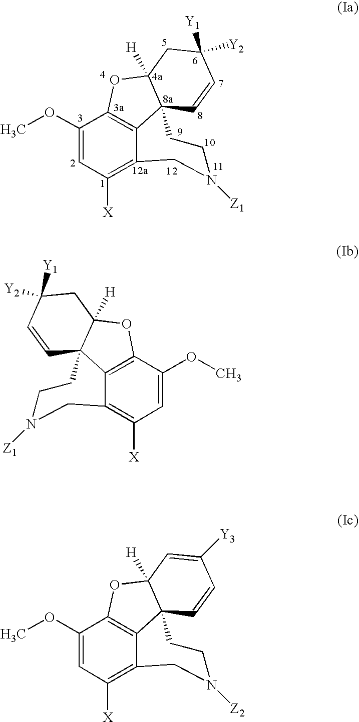

The invention relates to novel derivatives of 4a,5,9,10,11,12-hexahydro-benzofuro[3a,3,2] [2]benzazepine of general formulae 1a, 1b and 1c. Compounds 1a, 1b and 1c can be produced in an efficient manner on an industrial scale with the desired optical purity and are suitable for the production of medicaments for the treatment of a diverse range of symptoms, particularly diseases of the central nervous system (CNS), on account of the pharmacological effect thereof.

The invention concerns new derivatives of 4a,5,9,10,11,12-hexahydro-benzo-furo[3a,3,2][2] benzazepine, a method for producing them and their use in the production of drugs.

The compound type noted above also includes, among others, galanthamine derivatives. Galanthamine is a tetracyclic alkaloid that belongs to the group of the reversibly acting cholinesterase inhibitors and that is also used as an active agent in the treatment of Alzheimer's disease—see Neurologist 9, 235, 2003; Clinical Geriatrics 9(11), 55, 2001. Moreover, it is known from the literature that structural analogs of the naturally occurring galanthamine have different chemical properties—see Proc. Chem. Soc. 357, 1964. Thus, a change of the steric configuration of substituents on an asymmetric carbon atom leads to a significant change of the pharmacological properties—see Farmakol. Alkaloidov Serdech. Glikozidov 96, 1971, Russ. In particular the steric configuration at carbon 6 of the galanthamine parent substance is decisive with respect to pharmacological properties.

In spite of the fact that a number of methods for producing galanthamine are known, up to now it was not possible to produce optically active derivatives of the said 6-epi analogs of naturally occurring or synthetic galanthamine, since the optically active intermediate products needed for the synthesis, i.e., 11-demethyl-6-epigalanthamines, are not accessible. Chiral separations of 11-demethyl-galanthamine and 11-demethyl bromogalanthamine are described, for example, in WO-A-96/12692, WO-A-97/40049 and WO-A-01/74820. (−)-11-demethyl galanthamine can be obtained from a plant extract —see Nat. Prod. Sci. 4, 148, 1998—or synthetically (see U.S. PAT. NO. 5,958,903, WO-A-03/080623, WO-A-97/03987) from (−)-galanthamine. The recovery of a racemic mixture of a 1-bromine derivative of 11-demethyl epigalanthamine from a plant extract in the mLligram range is known only from Phytochemistry 34, 1656, 1993.

The invention is thus based on the task of making a contribution to the preparation of (+) and also (−)-11-demethyl-6-epigalanthamine that is also intended to enable an efficient use on an industrial scale.

In accordance with the invention, new derivatives of 4a,5,9,10,11,12-hexahydro-benzofuro[3a,3,2][2] benzazepine with the general formulas Ia or Ib 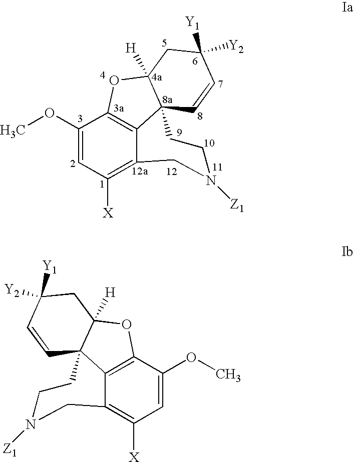
and their salts are proposed in accordance with the invention, where
Ia represents optically active (−) derivatives of galanthamine and Ib represents optically active (+) derivatives of galanthamine, which occur in a mirror configuration, and in which
Y₁ and Y₂ are alternately H or OH,
X=HorBrand
Z=a group of the following formulas 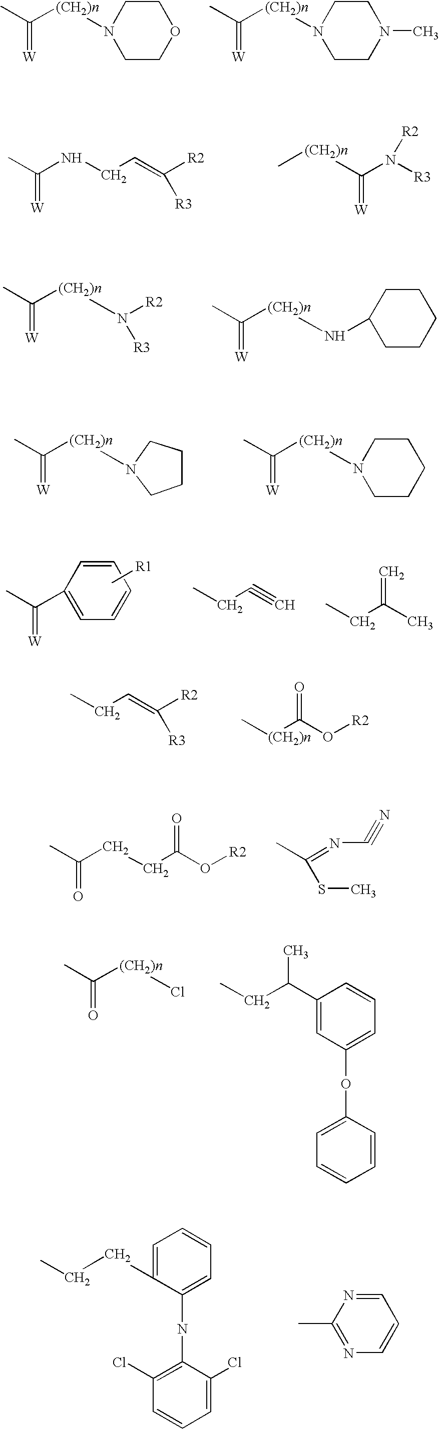 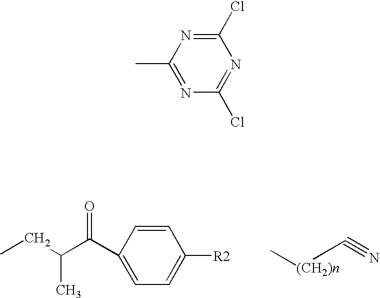
   in which
R₁=H, Cl, Br, i, F, OH, linear or branched (C₁-C₆) alkyl, linear or branched (C₁-C₆) alkyloxY₁ NO₂, NR₂R₃,
R₂=R₃=H, linear or branched (C₁-C₆) alkyl
W=H, O, S
n=0or 1-6
   and in which
Z₁ is equal to H solely for compounds 1, 3, 13 and 24 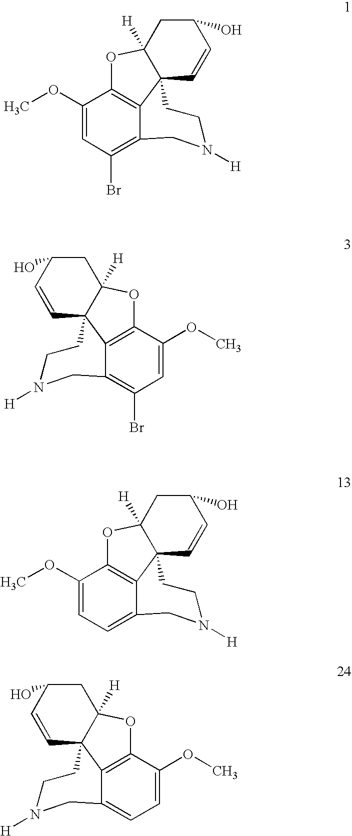
   where compounds 1 and 13 are (−) derivatives of 6-epinorgalanthamine and compounds 3 and 24 are (+) derivatives of 6-epinorgalanthamine, and in which
Z₁ is equal to hydroxypropyl solely for the compound 29 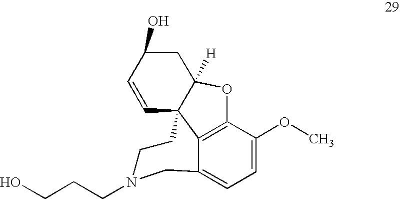
   and
Z₁ is equal to ethyl solely for the compound 26 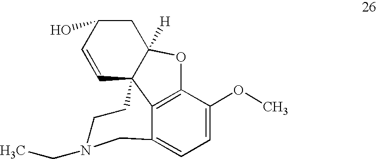
   and
Z₁ is equal to methyl solely for the following compounds 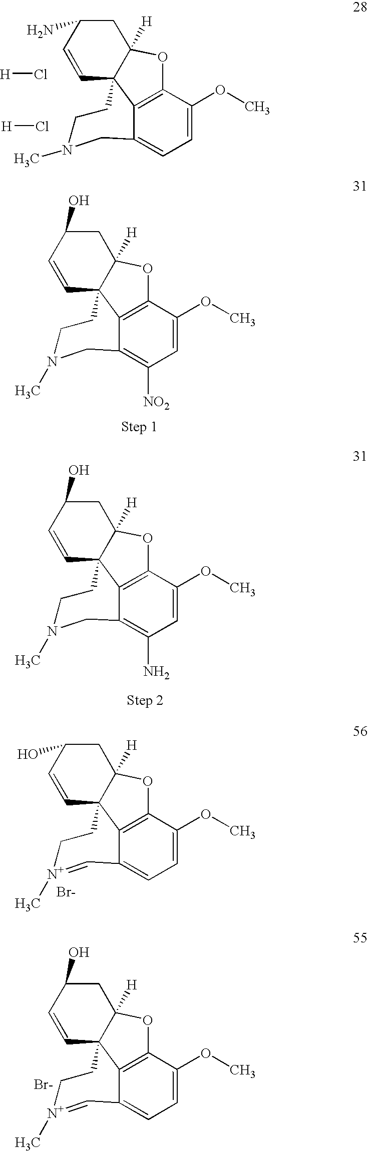
   and
   where compounds 29, 31 and 55 are (+) derivatives of galanthamine and compounds 26, 28 and 56 are (+)-epi derivatives of galanthamine.

In accordance with the invention compounds Ia and Ib are produced by converting natural and also synthetic 11-demethyl galanthamine to the corresponding 6-epi analogs. Through treatment with dilute acid, this method is also suitable for preparation of optically active derivatives of 11-demethyl-6-epigalanthamine, since only the configuration in position 6 is altered during the preparation, whereas the two other centers of asymmetry 4a and 8a remain unchanged.

Starting from these optically active starting materials the invention makes available an efficient and industrially applicable method for producing optically active derivatives of (−)-epigalanthamine and also the optically active (+)-epigalanthamine. Through the use of the invention not only can the (−) derivatives that occur in nature, but also the (+) derivatives of 4a,5,9,10,11,12-hexahydrobenzofuro[3a,3,2][2]benzazepine that do not occur in nature can be prepared synthetically.

The method in accordance with the invention has the advantage that the changes of configuration both in the case of the natural derivatives and those that do not occur in nature are carried out with the optically active analogs of galanthamine and not with the analogs of 6-epigalanthamine. All 4 derivatives, namely N-demethyl analoges of (−)-galanthamine, (+)-galanthamine and (−)-6-epigalanthamine and (+)-6-epigalanthamine can be prepared by this method after a single racemate separation.

The invention further concerns new derivatives of 4a,5,9,10,11,12-hexa-hydro-benzofuro[3a,3,2][2]benzazepine with the general formula Ic 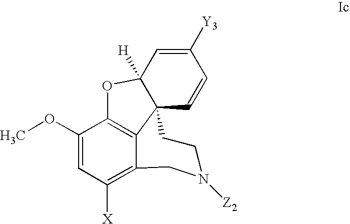
and their salts, in which
X is H or Br,
Z₂ is H, linear or branched (C₁-C₆) alkyl, linear or branched (C₂-C₇) alkenyl, linear or branched (C₂-C₇) alkinyl and
Y₃ is linear or branched (C₁-C₆) alkyl, phenyl, linear or branched (C₁-C₆) alkylphenyl, nitrophenyl, chlorophenyl, bromophenyl, aminophenyl, hydroxyphenyl.

The compounds of general formula Ic are also important to the extent that they exhibit pharmacological activitY₁ which can be seen from the following table, in which “AchE” means acetylcholinesterase, “BchE” means butyrylcholinesterase and IC₅₀ means the concentration at which 50% inhibition occurs.

| | | | Acetyl- | Butyryl- | |
|---|---|---|---|---|---|
| Example | | | cholinesterase | cholinesterase | |
| No. | Structure | stereo | IC-50 (μM) | IC-50 (μM) | Name |
| 1 | 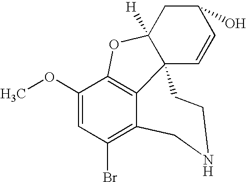 | (−) epi | >100 | >100 | (4aS,6S,8S)-1-bromo-4a,5,9,10,11,12-hexahydro- 3-methoxy-6H-[1]benzofuro[3a,3,2-ef][2]benzazepin- 6-ol. |
| 2 | 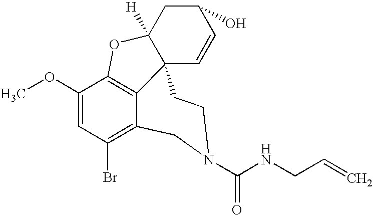 | (−) epi | 51 | >100 | (4aS,6S,8aS)-1-bromo-6-hydroxy-3-methoxy-4a,5, 9,10-tetrahydro-6N-[1]benzofuro[3a,3,2-et][2]benzazepine-11(12H)-thiocarbonic acid allylamide |
| 3 | 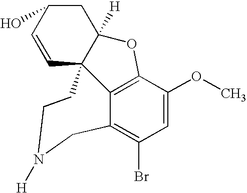 | (+) epi | >100 | >100 | (4aR,6R,8R)-1-bromo-4a,5,9,10,11,12- hexahydro-3-methoxy-6H-[1]benzofuro[3a,3,2-ef][2]benzazepin-6-ol. |
| 4 | 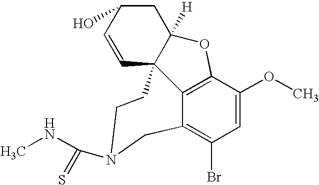 | (+) epi | >100 | >100 | (4aR,6R,8aR)-1-brorno-4a,5,9,10-tetrahydro- 6-hydroxy-3-methoxy-6H-[1]benzofuro-[3a,3,2-ef][2]benzazepine-11(12H)-thiocarbonic acid methylamide |
| 5 | 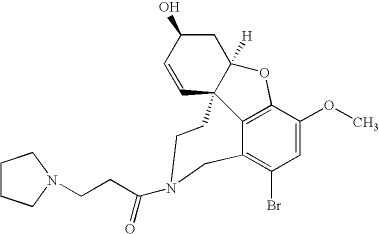 | (+) | >100 | 66 | 1-[(4aR,6S,8aR)-6-hydroxy-3-methoxy- 5,6,9,10-tetrahydro-4aH-[1]benzofuro[3a,3,2-ef][2]benzazepine-11(12H)-yl]-3-(1-pyrrolidyl) propan-1-one |
| 6 | 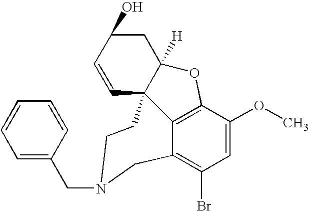 | (+) | >100 | 19 | (4aR,6S,8aR)-11-benzyl-1-bromo-4a,5,9,10,11,12- hexahydro-3-methoxy-6H-[1]benzofuro[3a,3,2-ef][2]benzazepin-6-ol |
| 7 | 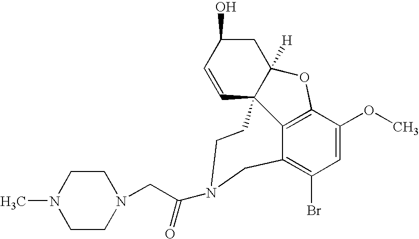 | (+) | 89 | >100 | 1-[(4aR,6S,8aR)-1-bromo-6-hydroxy-3- methoxy-5,6,9,10-tetrahydro-4aH- [1]benzofuro[3a,3,2-ef][2]benzazepine- 11(12H)-yl]-2-(4-methylpiperazinyl)ethan-1-one |
| 8 | 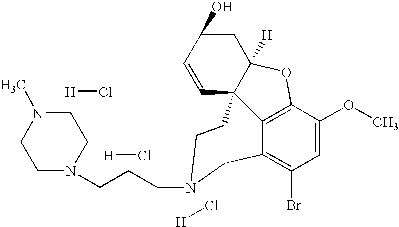 | (+) | >100 | 31 | (4aR,6R,8aR)-11-(3-(4-methylpiperazine)-1-yl- propyl)-3-methoxy-5,6,9,10,11,12-hexahydro-4aH- [1]benzofuro[3a,3,2-ef][2]benzazepin-6-oI, Trihydrochloride |
| 9 | 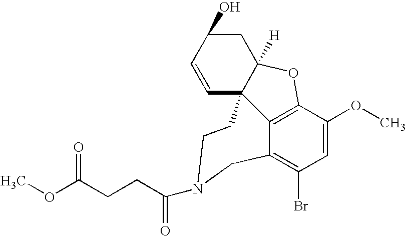 | (+) | >100 | >100 | Methyl-4-((4aR,6S,8aR)-1-bromo- 4a,5,9,10,11,12-hexahydro-6-hydroxy-3- methoxy-6H-benzofuro[3a,3,2-ef][2]benzazepine- 11-yl)gamma-oxobutyrate |
| 10 | 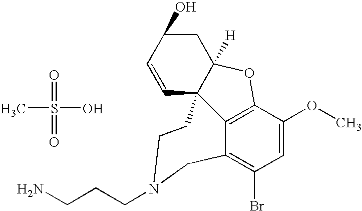 | (+) | >100 | >100 | (4aR,6S,8aR)-11-(4-aminopropyl)-3- methoxy-5,6,9,10,11,12-hexahydro-4aH- [1]benzofuro[3a,3,2-et][2]benzazepin-6-ol, methane sulfonate |
| 11 | 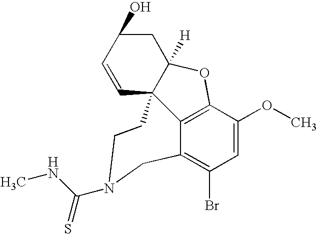 | (+) | >100 | >100 | (4aR,6S,8aR)-1-bromo-4a,5,9,10-tetrahydro-6- hydroxy-3-methoxy-6H-[1]benzofuro-[3a,3,2-et][2]benzazepine-11(12H)-thiocarbonic acid methylamide |
| 12 | 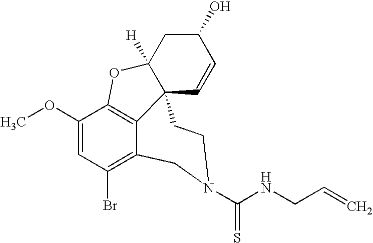 | (−) | >100 | 11 | (4aS,6R,8aS)-1-bromo-6-hydroxy-3-methoxy- 4a,5,9,10-tetrahydro-6H-[1]benzofuro[3a,3,2-ef][2]benzazepine-11(12H)-thiocarbonic acid allylamide |
| 13 | 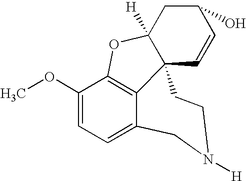 | (−) epi | 15 | 0.56 | (4aS,6S,8aS)-4a,5,9,10,11,12-hexahydro-3- methoxy-6H-[1]benzofura[3a,3,2-ef][2]benzazepin- 6-ol |
| 14 | 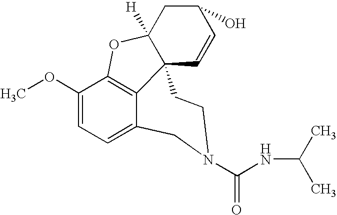 | (−) epi | >100 | >100 | (4aS,6S,8aS)-6-hydroxy-3-methoxy-4a,5,9,10- tetrahydro-6H-[1]benzofuro[3a,3,2-ef][2]benzazepine-11(12H)-thiocarbonic acid methylamide |
| 15 | 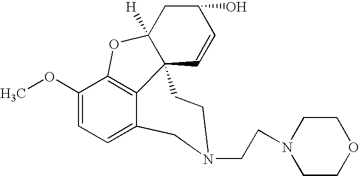 | (−) epi | 84 | >100 | (4aS,6S,8aS)-4a,5,9,10,11,12-hexahydro-11- (2-(morpholin-4-yl)-ethyl)-3-methoxy-6H- [1]benzofuro[3a,3,2-ef][2]benzazepin-6-ol |
| 16 | 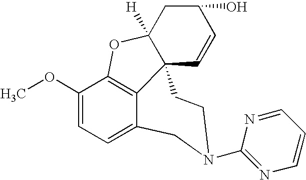 | (−) epi | >100 | >100 | (4aS,6S,8aS)-4a,5,9,10,11,12-hexahydro-3- methoxy-11-(2-pyrimidinyl)-6H- [1]benzofuro[3a,3,2-ef][2]benzazepin-6-ol |
| 17 | 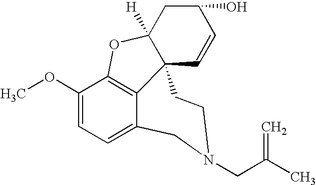 | (−) epi | >100 | >100 | (4aS,6S,SaS)-4a,5,9,10,11,12-hexahydro-3- methoxy-11-(2-methyl-prop-2-enyl)-6H- [1]benzofuro[3a,3,2-ef][2]benzazepin-6-ol |
| 18 | 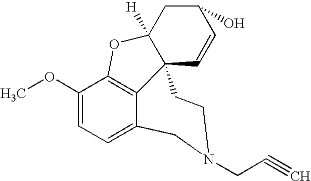 | (−) epi | >100 | >100 | (4aS,6S,8aS)-4a,5,9,10,11,12-hexahydro-3- methoxy-11-propargyl-6H-[1]benzofuro[3a,3,2-ef][2]benzazepin-6-ol |
| 19 | 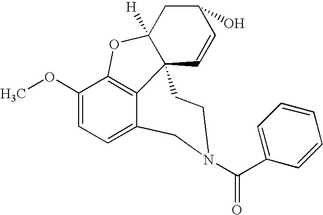 | (−) epi | >100 | >100 | (4aS,6S,8aS)-4a,5,9,10,11,12-hexahydro-3- methoxy-11-benzoyl-6H-[1]benzafuro[3a,3,2-ef][2]benzazepin-6-ol |
| 20 | 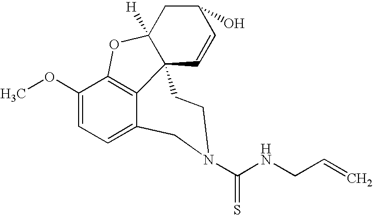 | (−) epi | >100 | >100 | (4aS,6S,8aS)-6-hydroxy-3-methoxy-4a,5,9,10- tetrahydro-6H-[1]benzofuro[3a,3,2-ef][2]benzazepine-11(12H)-thiocarbonic acid allylamide |
| 21 | 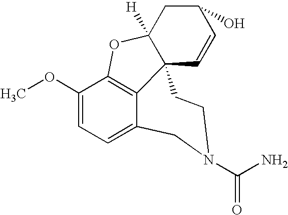 | (−) epi | >100 | >100 | (4aS,6S,8aS)-4a,5,9,10-tetrahydro-6-hydroxy-3- methoxy-6H-[1]benzofuro[3a,3,2-ef][2]benzazepine-11(12H)-carboxamide |
| 22 | 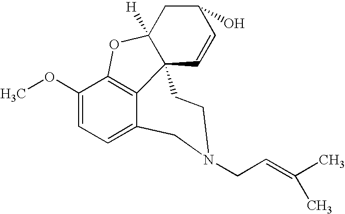 | (−) epi | 6 | 20 | (4aS,6S,8aS)-4a,5,9,10,11,12-hexahydro-3- methoxy-11-(3-Methylbut-2-en-1-yl)-6H-[1]benzofuro[3a,3,2-ef][2]benzazepin-6-ol |
| 23 | 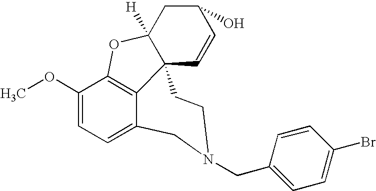 | (−) epi | 49 | 10 | (4aS,6S,8aS)-1-bromo-11-(4-brombenzyl)- 4a,5,9,10,11,12-hexahydro-3-methoxy-6H- [1]benzofuro[3a,3,2-ef][2]benzazepin-6-ol |
| 24 | 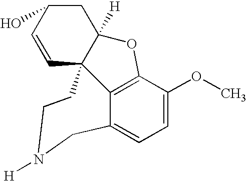 | (+) epi | >100 | >100 | (4aR,6R,8R)-4a,5,9,10,11,12-hexahydro-3- methoxy-6H-[1]benzofuro[3a,3,2-ef][2]benzazepin-6-ol, |
| 25 | 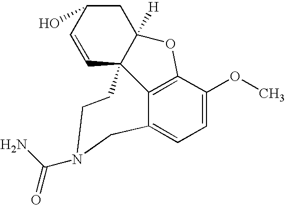 | (+) epi | >100 | >100 | (4aR,6R,8aR)-4a,5,9,10-tetrahydro-6-hydroxy-3- methoxy-6H-[1]benzofuro[3a,3,2-ef][2]benzazepine-11(12H)-carboxamide |
| 26 | 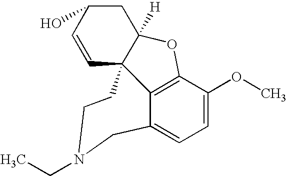 | (+) epi | >100 | >100 | (4aR,6R,8aR)-4a,5,9,10,11,12-hexahydro-3- methoxy-11-ethyl-6H-[1]benzofuro[3a,3,2,ef][2]benzazepin-6-ol |
| 27 | 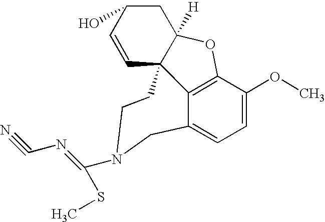 | (+) epi | >100 | >100 | Methyl (4aR,6R,8aR)-N11-cyano-6-hydroxy-3- methoxy-4a,5,9,10-tetrahydro-6H-[1]benzofuro [3a,3,2-ef][2]benzazepine-11(12H)- carboximidothioate |
| 28 | 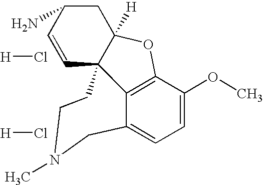 | (+) epi | >100 | >100 | (4aR,6R,8aR)-4a,5,9,10,11,12-hexahydro-3- methoxy-11-methyl-6H-benzofuro[3a,3,2-ef][2]benzazepin-6-amine, dihydrochloride |
| 29 | 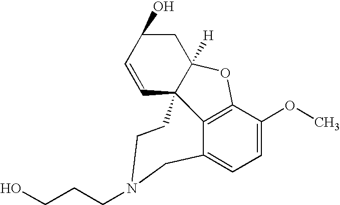 | (+) | >100 | >100 | (4aR,6S,8aR)-11-(3-hydroxypropyl)-3- methoxy-5,6,9,10,11,12-hexahydro-4aH- [1]benzofuro[3a,3,2-ef][2]benzazepin-6-ol |
| 30 | 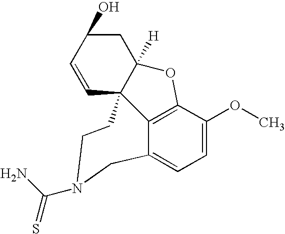 | (+) | >100 | >100 | (4aS,6R,8aS)-6-hydroxy-3-methoxy-5,6,9,10- tetrahydro-4aH-[1]benzofuro[3a,3,2-ef][2]benzazepine-11(12H)-carbothioamide |
| 31 | 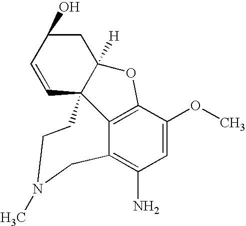 | (+) | >100 | >100 | (4aS,6S,8aS)-4a,5,9,10,11,12-hexahydro-3- methoxy-11-benzoyl-6H-[1]benzofuro[3a,3,2-ef][2]benzazepin-6-ol |
| 32 | 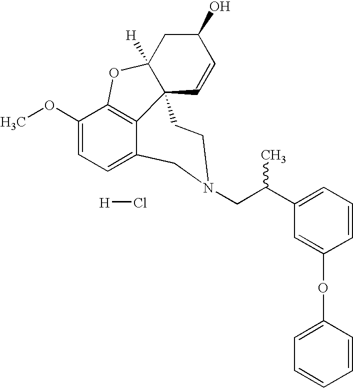 | (−) | 0.1 | 0.36 | 2-((4aS,6R,8aS)-6-hydroxy-3-methoxy-5,6,9,10- tetrahydro-4aH-[1]benzofuro[3a,3,2-ef][2]benzazepine-11(12H)-yl)-1-methyl-1- (3-phenoxyphenyl)ethane hydrochloride |
| 33 | 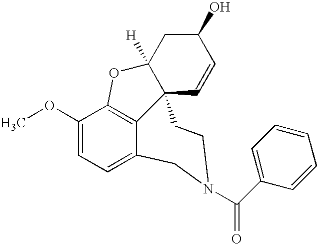 | (−) | 27 | >100 | (4aS,6R,8aS)-4a,5,9,10,11,12-hexahydro-3- methoxy-11-benzoyl-6H-[1]benzofuro[3a,3,2-ef][2]benzazepin-6-ol |
| 34 | 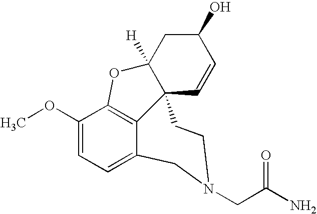 | (−) | >100 | 78 | 2-((4aS,6R,8aS)-6-hydroxy-3-methoxy-5,6,9,10- tetrahydro-4aH-[1]benzofuro[3a,3,2-ef][2]benzazepine-11(12H)-yl)acetamide |
| 35 | 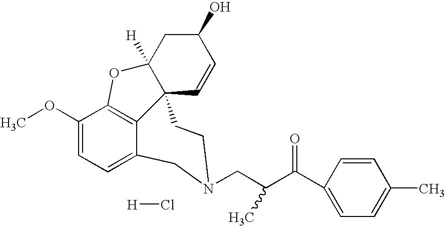 | (−) | 0.19 | 0.4 | 3-[(4aS,6R,8aS)-6-hydroxy-3-methoxy-5,6,9,10- tetrahydro-4aH-[1]benzofuro[3a,3,2-ef][2]benzazepine-11(12H)-yl]-2-methyl-1(- 4-methylphenyl)propan-1-one, hydrochloride |
| 36 | 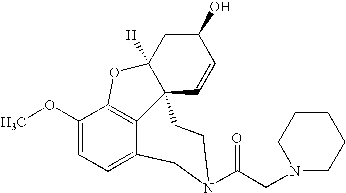 | (−) | 11 | >100 | 1-[(4aS,6R,8aS)-6-hydroxy-3-methoxy-5,6,9,10- tetrahydro-4aH-[1]benzofuro[3a,3,2-ef][2]benzazepine-11(12H)-yl]-2-(1-piperidyl)ethan- 1-one |
| 37 | 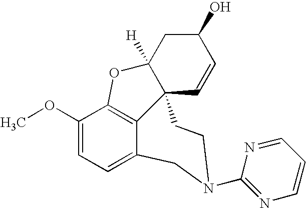 | (−) | >100 | >100 | (4aS,6R,8aS)-3-methoxy-11-(2-pyrimidinyl)- 5,6,9,10,11,12-hexahydro-4aH-[1]benzofuro [3a,3,2-ef][2]benzazepin-6-ol |
| 38 | 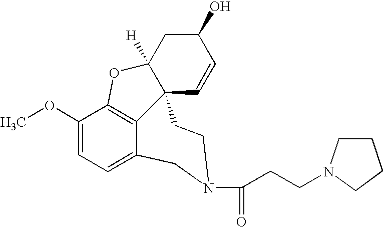 | (−) | 5.3 | 2.4 | 1-[(4aS,6R,8aS)-6-hydroxy-3-methoxy-5,6,9,10- tetrahydro-4aH-[1]benzofuro[3a,3,2-ef][2]benzazepine-11(12H)-yl]-3-(1-pyrrolidyl) propan-1-one |
| 39 | 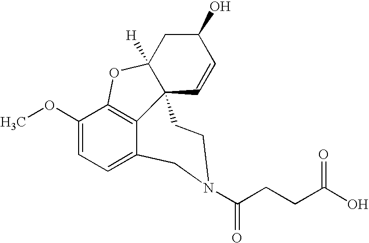 | (−) | >100 | >100 | ((4aS,6R,8aS)-4a,5,9,10,11,12-hexahydro-6- hydroxy-3-methoxy-6H-benzofuro[3a,3,2-ef][2]benzazepine-11--yl)gamma-oxobutyric acid |
| 40 | 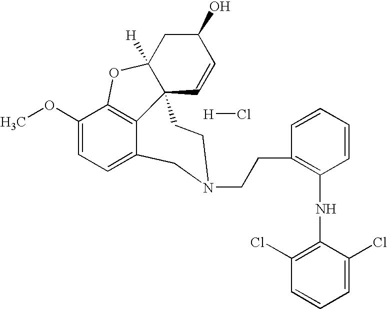 | (−) | 1.1 | 1.3 | 1-((4aS,6R,8aS)-6-hydroxy-3-methoxy-5,6,9,10- tetrahydro-4aH-[1]benzofuro[3a,3,2-ef][2]benzazepine-11(12H)-yl)-2-[2-(2,6-dichloranilino)]- phenylethane |
| 41 | 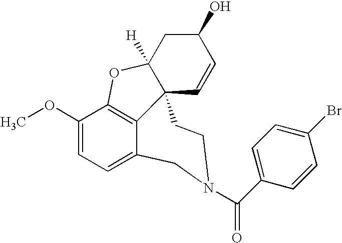 | (−) | 3.9 | 30 | (4aS,6R,8aS)-4a,5,9,10,11,12-hexahydro-3- methoxy-11-(4-bromo-benzoyl)-6H-[1]benzofuro[3a,3,2-ef][2]benzazepin-6-ol |
| 42 | 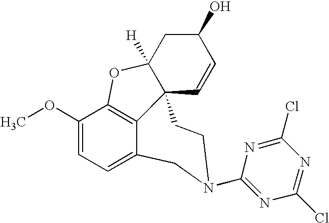 | (−) | >100 | >100 | (4aS,6R,8aS)-11-(4,6-dichloro-1,3,5,-triazin-2-yl)- 3-methoxy-5,6,9,10,11,12-hexahydro-4aH- [1]benzofuro[3a,3,2-ef][2]benzazepin-6-ol |
| 43 | 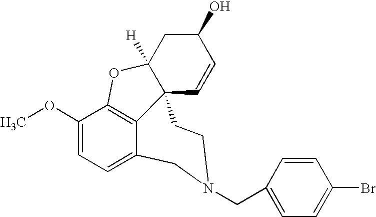 | (−) | 0.016 | 0.0006 | (4aS,6R,8aS)-11-(4-brombenzyl)-4a,5,9,10,11,12- hexahydro-3-methoxy-6H-[1]benzofuro[3a,3,2-ef][2]benzazepin-6-ol |
| 44 | 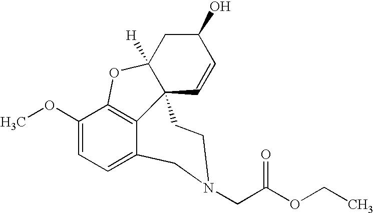 | (−) | 8.8 | 42 | Ethyl-2-((4aS,6R,8aS)-6-hydroxy-3-methoxy- 5,6,9,10-tetrahydro-4aH-[1]benzofuro[3a,3,2-ef][2]benzazepine-11(12H)-yl)acetate |
| 45 | 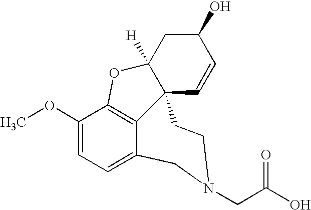 | (−) | | | 2-((4aS,6R,8aS)-6-hydroxy-3-methoxy-5,6,9,10- tetrahydro-4aH-[1]benzofuro[3a,3,2-ef][2]benzazepine-11(12H)-yl)acetic add |
| 46 | 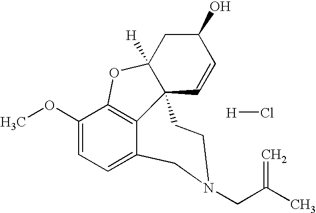 | (−) | 1.1 | 0.34 | (4aS,6R,8aS)-4a,5,9,10,11,12-hexahydro-3- methoxy-11-(2-methyl-prop-2-enyl)-6H-[1]benzofuro[3a,3,2-ef][2]benzazepin-6-ol, hydrochloride |
| 47 | 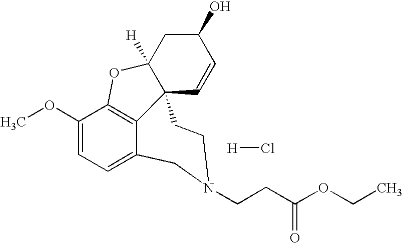 | (−) | 2.6 | 51 | Ethyl-3-((4aS,6R,8aS)-6-hydroxy-3-methoxy- 5,6,9,10-tetrahydro-4aH-[1]benzofuro[3a,3,2-ef][2]benzazepine-11(12H)-yl)propanoate, hydrochloride |
| 48 | 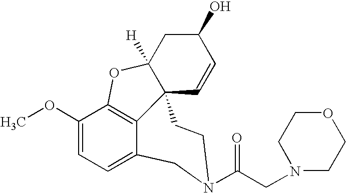 | (−) | 4.9 | 3.8 | 1-[(4aS,6R,8aS)-6-hydroxy-3-methoxy-5,6,9,10- tetrahydro-4aH-[1]benzofuro[3a,3,2-ef][2]benzazepine-11(12H)-yl]-2-(4-morpholinyl) ethan-1-one |
| 49 | 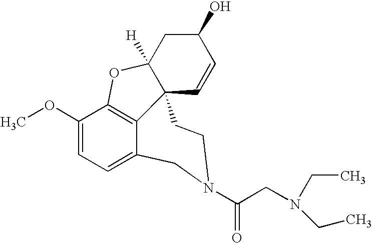 | (−) | 5.6 | 40 | 1-((4aS,6R,8aS)-6-hydroxy-3-methoxy-5,6,9,10- tetrahydro-4aH-[1]benzofuro[3a,3,2-ef][2]benzazepine-11(12H)-yl]-2-(diethylamino) ethan-1-one |
| 50 | 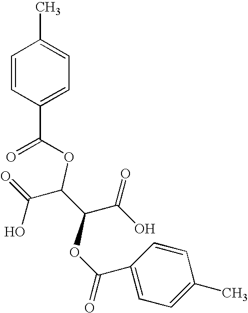 | (−) | 0.036 | 0.61 | (4aS,6R,8aS)-4a,5,9,10,11,12-hexahydro-3- methoxy-11-[3-(1-piperidinyl)butyl]-6H- benzofuro[3a,3,2-ef][2]benzazepin-6-ol, (+) Di-O-p-toluoyl tartrate, |
| | 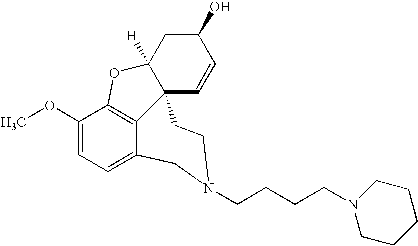 | | | | |
| 51 | 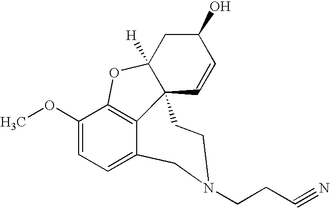 | (−) | 33 | 57 | 3-((4aS,6R,8aS)-1-bromo-6-hydroxy-3-methoxy-5, 6,9,10-tetrahydro-4aH-[1]benzofuro[3a,3,2-ef][2]benzazepine-11(12H)-yl)propanenitrile |
| 52 | 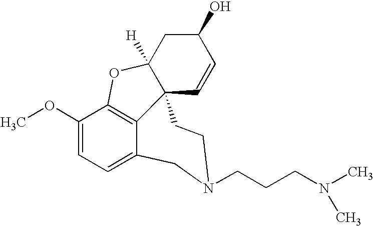 | (−) | 1.3 | 2.1 | (4aS,6R,8aS)-11-((3-dimethylamino)propyl)-3- methoxy-5,6,9,10,11,12-hexahydro-4aH-[1]benzofuro[3a,3,2-ef][2]benzazepin-6-ol |
| 53 | 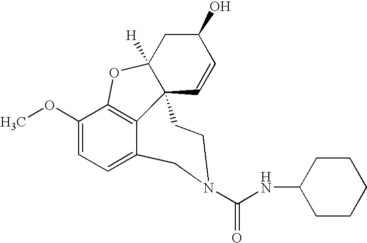 | (−) | 1.3 | >100 | (4aS,6R,8aS)-N11-cydohexyl-6-hydroxy-3- methoxy-5,6,9,10-tetrahydro-4aH-[1]benzofuro[3a, 3,2-ef][2]benzazepine-11(12H)-carbonic acid isopropylamide |
| 54 | 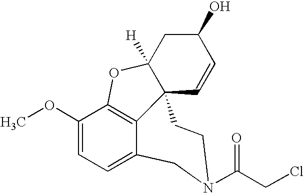 | (−) | 51 | >100 | 1-[(4aS,6R,8aS)-6-hydroxy-3-methoxy-5,6,9,10- tetrahydro-4aH-[1]benzofuro[3a,3,2-ef][2]benzazepine-11(12H)-yl]-2-chloroethan-1-one |
| 55 | 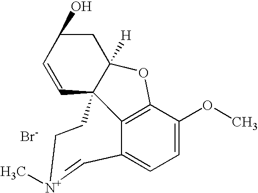 | (+) | >100 | >100 | (4aR,6S,8aR)-6-hydroxy-3-methoxy-11-methyl- 4a,5,9,10-tetrnhydro-6H-benzofuro[3a,3,2-eq][2]benzazepinium bromide |
| 56 | 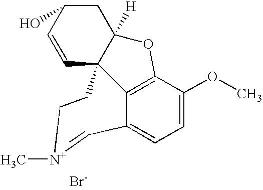 | (+) epi | >100 | >100 | (4aR,6R,8aR)-6-hydroxy-3-methoxy-11- methyl-4a,5,9,10-tetrahydro-6H-benzofuro[3a,3, 2-ef][2]benzazepineium bromide |
| 57 | 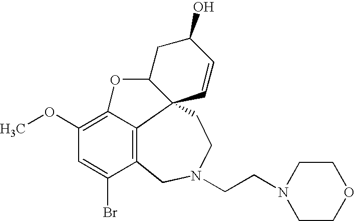 | (−) | >100 | 2.7 | (4aS,6R,8aS)-1-bromo-4a,5,9,10,11,12- hexahydro-11-(2-(mopholin-4-yl)-ethyl)-3- methoxy-6H-[1]benzafuro[3a,3,2-ef][2]benzazepin-6-ol |
| 58 | 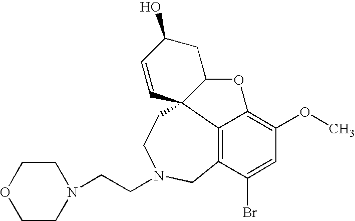 | (+) | >100 | 53 | (4aR,6R,8aRS)-1-bromo-4a,5,9,10,11,12- hexahydro-11-(2-(morpholin-4-yl)-ethyl)-3- methoxy-6H-[1]benzofuro[3a,3,2-ef][2]benzazepin-6-ol |
| 59 | 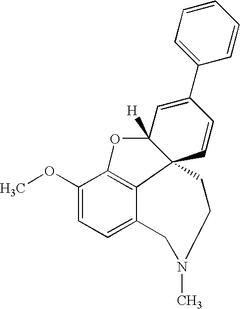 | (−) | 52 | 25 | (4aS,8aS)-D5,6-4a,5,9,10,11,12-hexahydro-11- methyl-3-methoxy-6-phenyl-6H-[1]benzofuro[3a,3, 2-ef][2]benzazepine |
| 60 | 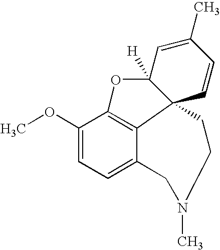 | (−) | 80 | 200 | (4aS,8aS)-D5,6--4a,5,9,10,11,12-hexahydro-6,11- dimethyl-3-methoxy-6H-[1]benzofuro[3a,3,2-ef][2]benzazepine |
| 61 | 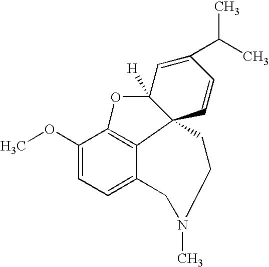 | (−) | >100 | 9 | (4aS,8aS)-D5,6-4a,5,9,10,11,12-hexahydro-6- (isopropyl)-11-methyl-3-methoxy-6H-[1]benzofuro [3a,3,2-ef][2]benzazepine |

The pharmacological activity of compounds Ia, Ib and Ic in accordance with the invention can be shown by means of the IC₅₀ values.

AccordinglY₁ the invention also concerns drugs that contain one or more of the compounds Ia, Ib or Ic in accordance with the invention as pharmaceutical active agents.

The invention additionally concerns the use of one or more compounds Ia, Ib or Ic in pure form or in the form of their pharmaceutically safe acid addition salts to produce a drug for the treatment of Alzheimer's, disease and related conditions of dementia, for the treatment of Parkinson's disease, Huntington's disease (chorea), for the treatment of multiple sclerosis or amyotrophic lateral sclerosis, for the treatment of epilepsY₁ for the treatment of effects of a stroke or craniocerebral trauma, for the treatment and prophylaxis of the effects of diffused oxygen and nutrient deficiency in the brain such as are observed after hypoxia, anoxia, asphyxia, cardiac arrest, intoxications, narcosis and in the infant after complications in cases of difficult birth, for the prophylactic treatment of apoptotic degeneration in neurons, which have been or are being damaged by local radio- or chemotherapy of brain tumors.

The invention additionally concerns the use of one or more compounds Ia, Ib or Ic in pure form or in the form of their pharmaceutically safe acid addition salts to produce a drug for the treatment of bacterial meningitis, for the treatment of diseases within an apoptotic component, especially in the wake of amyloid-associated cell degeneration and for the treatment of diabetes mellitus, especially when the disease is accompanied by amyloid degeneration of the islet cells.

The invention further concerns the use of one or more compounds Ia, Ib or Ic in pure form or in the form of their pharmaceutically safe acid addition salts to produce a drug for the treatment or preventative of postoperative delirium and/or subsyndromal postoperative delirium.

The following examples show possible synthesis paths to the preparation of compounds Ia, Ib and Ic in accordance with the invention:

### EXAMPLE 1

### (4aS,6S,8S)-1-Bromo-4a,5,9,10,11,12-hexahydro-3-methoxy-6H-[1]benzofuro[3a,3,2-ef][2]benzazepin-6-ol, (Ia Y₁=H, Y₂=OH, X=Br, Z₁=H)

20 g (−)-bromonorgalanthamine, prepared in accordance with WO-A-97/40049, is stirred in 800 mL 2% HCl solution at the boiling point with reflux cooling. After 3 h the reaction mixture is cooled, made basic with ammonia solution and extracted with 3×300 mL chloroform. The combined organic phases are dried over sodium sulfate. The drying agent is filtered out and the filtrate is vacuum concentrated.

Yield: 14.9 g (75% of theory) M.p.: 198-203° C. R_{f}: 0.25 (chloroform:MeOH:ammonia solution=90:9:1)

¹H-NMR (CDCl₃): δ 6.85 (s, 1H), 6.10 (d, 1H), 5.82 (d, 1H), 4.59 (m, 2H), 4.49 (d, 1H), 3.83 (d,lH), 3.80 (s, 3H), 3.30 (dd, 1H), 3.22 (dt, 1H), 2.72 (d, 1H), 1.88 (m, 2H), 1.71 (t, 1H); ATP-NMR (CDCl₃): 6146.8 (s) 143.9 (s) 134.2 (s), 132.4 (d), 131.5 (s), 126.3 (d), 115.4 (d), 112.5 (s), 88.6 (d), 62.7 (d), 56.2 (q), 52.4 (t), 49.2 (s), 46.9 (t) 40.6 (t), 32.1 (t);

### EXAMPLE 2

### (4aS,6S,8aS)-1-Bromo-6-hydroxy-3-methoxy-4a,5,9,10-tetrahydro-6H-[1 ]benzofuro[3a, 3,2-ef][2]benzazepine-11(12H)-thiocarbonic acid allylamide (Ia Y₁=H, Y₂=OH, X=Br, Z₁=C₄H₅NS)

2.0 g (5.6 mmol) (−)-epibromonorgalanthamine (Ia, Y₁=H, Y₂=OH, X=Br, Z₁=H) is dissolved in 60 mL tetrahydrofuran, mixed with 0.6 mL allyl isothiocyanate and stirred at 60° C. under reflux cooling. After 40 h the solvent is vacuum distilled out and the residue is crystallized from chloroform/n-hexane.

Yield: 2.28 g (76% of theory) M.p.: 199-207° C. R_{f}: 0.75 (chloroform:MeOH=9:1)

¹H-NMR (CDCI₃): δ 6.88 (s, 1H), 6.05 (d, 1H), 5.90 (m, 2H), 5.53 (d, 1H), 5.19 (d,lH), 5.10 (d, 1H), 4.64 (b, 2H), 4.50 (d, 1H), 4.31 (m, 1H), 4.19 (m,lH), 3.88 (s, 3H), 3.55 (t, 1H), 2.77 (m, 1H), 2.30 (t, 1H), 2.08 (b, 1H), 1.92 (d, 1H), 1.78(dt, 1H); ATP-NMR (CDCl₃): δ 180.8 (s), 148.2 (s) 145.7 (s) 134.3 (s), 133.9 (d), 133.5 (d), 125.7 (d), 125.3 (s), 117.9 (s), 115.4 (d), 112.3 (s), 89.0 (d), 63.3 (d), 56.7 (q), 52.0 (d), 49.5 (2t), 49.3 (s), 36.8 (t), 32.3 (t);

### EXAMPLE 3

### (4aR,6R,8aR)-1-Bromo-4a,5,9,10,11,12-hexahydro-3-methoxy-6H-[ 1]benzofuro[3a,3,2-ef][2]benzazepin-6-ol (Ib Y₁=H, Y₂=OH, X=Br, Z₁=H)

20 g (+)-bromonorgalanthamine, prepared in accordance with WO-A-97/40049, is stirred in 800 mL 2% HCl solution at the boiling point under reflux cooling. After 3 h the reaction mixture is cooled, made basic with cc ammonia solution and extracted with 3×300 mL chloroformn. The combined organic phases are dried over sodium sulfate. The drying agent is filtered out and the filtrate is vacuum concentrated.

Yield: 15.5 g (78% of theory) M.p.: 103-205° C.

R_{f}: 0.25 (chloroform:MeOH: ammonia solution=90:9:1)

¹H-NMR (CDCl₃): δ 6.88 (s, 1H), 6.07 (d, 1H), 5.82 (d, 1H), 4.59 (m, 2H), 4.51 (d, 1H), 3.83 (d,lH), 3.80 (s, 3H), 3.28 (d, 1H), 3.22 (t, 1H), 2.78 (d, 1H), 1.91 (m, 2H), 1.73 (t, 1H); ATP-NMR (CDCl₃): δ 146.8 (s) 143.9 (s) 134.2 (s), 132.4 (d), 131.5 (s), 126.3 (d), 115.4 (d), 112.5 (s), 88.6 (d), 62.7 (d), 56.2 (q), 52.4 (t), 49.2 (s), 46.9 (t) 40.6 (t), 32.1 (t);

### EXAMPLE 4

### (4aR,6R,8aR)-1-Bromo-4a,5,9,10-tetrahydro-6-hydroxy-3-methoxy-6H-[1 ]benzofuro-[3a,3,2-ef][2]benzazepine-11(12H)-thiocarbonic acid methylamide (Ib, Y₁=H, Y₂=OH, X=Br, Z₁=C₂H4NS)

1.96 g (+)-epibromonorgalanthamine (Ib Y₁=H, Y₂=OH, X=Br, Z₁=H) and 0.7 g methyl isothiocyanate are stirred in 50 mL toluene at the reflux temperature. After 16 h the reaction mixture is cooled to room temperature, the solvent is vacuum distilled out, and the residue is mixed with 200 mL 2N HCl and with 50 mL ethyl acetate. After separating the organic phase the aqueous phase is extracted with 2×50 mL ethyl acetate. The combined organic phases are dried over sodium sulfate, filtered, and vacuum concentrated. The product is purified by column chromatography (chloroform:MeOH=95:5).

Yield: 1.4 g (54% of theory) M.p.: 80-88° C. R_{f}: 0.45 (chloroform:MeOH:ammonia solution-90:9:1)

¹H-NMR (DMSO): δ 8.35 (b, 1H, N-H), 6.99 (s, 1H), 6.01 (b, 1H), 5.72 (d, 1H), 4.98 (d, 1H), 4.61 (b, 1H), 4.29 (m, 1H), 3.79 (d,lH), 3.74 (s, 3H), 2.90 (d, 3H), 2.51 (d, 1H), 2.48 (t, 1H), 1.98 (t, 1H), 1.81 (m, 2H), 1.65 (t, 1H); ATP-NMR (DMSO): δ 182.0 (s), 147.7 (s) 144.6 (s) 134.4 (s), 133.8 (d), 128.6 (s), 126.8 (d), 116.4 (d), 113.5 (s), 88.5 (d), 62.0 (d), 56.8 (q), 49.2 (t), 40.0 (s), 36.5 (t) 33.7 (q), 32.4 (t);

### EXAMPLE 5

### 1-[(4aR,6S,8aR)-6-Hydroxy-3-methoxy-5,6,9, 10-tetrahydro-4aH-[ 1]benzofuro[3a,3,2-ef][2]benzazepine-11(12H)-yl]-3-(1-pyrrolidyl)propan-1-one (Ib Y₁=OH, Y₂=H, X=Br, Z₁=C₇H₁₂NO)

2.0 g (+)-bromonorgalanthamine, prepared in accordance with WO-A-97/40049, and 1.0 mL triethylamine and 0.6 mL 3-bromopropionyl chloride are stirred in 100 mL tetrahydrofuran at 0° C. After 10 min the reaction mixture is mixed with 1.6 g potassium carbonate and 0.6 mL pyrrolidine and stirring is continued at 90° C. After 17 h the solvent is distilled out and the residue is mixed with 50 mL water and 50 mL chloroform. After separating the organic phase the aqueous phase is extracted with 2×50 mL chloroform. The combined organic phases are dried over sodium sulfate, filtered, and vacuum concentrated. The product is purified by column chromatography (chloroform:MeOH:ammonia solution=90:9:1).

Yield: 1.88 g (69.3% of theory) M.p.: 80-85° C. R_{f}: 0.4 (chloroform:MeOH:ammonia solution=90:9:1)

¹H-NMR (CDCl₃): δ 6.90 (s, 1H), 6.07 (dd, 1H), 5.18 (d, 1H), 4.59 (m, 2H), 4.31 (d, 1H), 4.18 (m,1H), 3.80 (s, 3H), 3.78 (d, 1H), 3.22 (t, 1H), 2.90 (m, 3H), 2.68 (m, 3H), 2.6-2.35 (m, 8H), 2.01 (dd, 1H), 1.89 (dt, 1H); ATP-NMR (CDCl₃): δ 171.4 (s), 146.5 (s), 144.9 (s), 133.6 (s), 128.8 (d), 127.3 (s), 126.0 (d), 115.7 (d), 112.8 (s), 88.3 (d), 61.6 (d), 56.2 (q), 54.2 (2t), 51.8 (t), 51.4 (t) 49.0 (s), 44.5 (t), 35.6 (t), 33.0 (t), 29.6 (t) 23.4 (2t);

### EXAMPLE 6

### 4aR,6S,8aR)-11-Benzyl-1-bromo-4a,5,9,10,11,12-hexahydro-3-methoxy-6H-[1]benzofuro[3a,3,2-ef][2]benzazepine-6-ol (Ib Y₁=OH, Y₂=H, X=Br, Z₁=C₇H₇)

2.0 g (+)-bromonorgalanthamine, prepared in accordance with WO-A-97/40049, and 4.0 g potassium carbonate and 0.71 mL benzyl bromide are stirred at reflux temperature in 40 mL acetonitrile. After 3 h the reaction mixture is cooled to room temperature, the solvent is vacuum distilled out, and the residue is mixed with 60 mL water and 50 mL ethyl acetate. After separating the organic phase the aqueous phase is extracted two times with 2×50 mL ethyl acetate. The combined organic phases are dried over sodium sulfate, filtered, and vacuum concentrated. The product is purified by column chromatography (chloroform:MeOH=99:1).

Yield: 1.76 g yellow oil (69.8% of theory) R_{f}:=0.75 (chloroform:MeOH=:99:1)

¹H-NMR (DMSO): δ 7.28 (m, 5H), 6.92 (s, 1H), 6.18 (d, 1H), 5.85 (dd, 1H), 4.59 (b, 1H), 4.35 (d, 1H), 4.12 (m, 2H), 3.78 (s, 3H), 3.64 (d, 1H), 3.55 (d, 1H), 2.98 (d, 1H), 2.52 (s, 2H), 2.27 (d, 1H), 2.09 (m, 2H); ATP-NMR (DMSO): δ 146.9 (s), 144.6 (s), 139.7 (s) 135.0 (s), 129.5 (d), 129.5 (2d), 129.0 (2d), 128.7 (s), 127.7 (d), 127.4 (d), 116.3 (d), 113.3 (s), 87.7 (d), 60.5 (d), 56.7 (q), 56.4(t) 51.2 (t), 49.3 (s), 39.9 (t) 34.2 (t), 31.6 (t);

### EXAMPLE 7

### 1-[(4aR,6S,8aR)-1-Bromo-6-hydroxy-3-methoxy-5,6,9,10-tetrahydro-4aH-[ 1 ]benzofuro [3a,3,2-ef] [2]benzazepine-11(12H)-yl]-2-(4-methylpiperazinyl)ethan-1-one (Ib Y=OH, Y₂=H, X=Br, Z₁=C₇H₁₃N₂0)

2.0 g (+)-bromonorgalanthamine, prepared in accordance with WO-A-97/40049, and 3.92 g potassium carbonate are stirred in 50 mL tetrahydrofuran and the suspension is cooled to 0° C. using an ice bath. After adding 0.48 mL chloroacetyl chloride by drops the mixture is stirred another 30 min at 0° C. and then 1.4 mL N-methyl piperazine is added. After 48 h at reflux the mixture is allowed to cool, 150 mL water is added, and the mixture is extracted by shaking with 3×40 mL ethyl acetate. The combined organic phases are dried over sodium sulfate and concentrated. The residue is purified by column chromatography (chloroform:MeOH:ammonia solution=95:4.5:0.5).

Yield: 0.7 g white foam (17.9% of theory) R_{f}: 0.42 (chloroform:MeOH:ammonia solution=90:9:1)

¹H-NMR (CDCl₃): δ 6.88 (s, 1H), 6.05 (dd, 1H), 5.94 (d, 1H), 5.59 (d, 1H), 4.58 (b, 1H), 4.31 (d, 1H), 4.12 (t, 1H), 3.85 (s, 3H), 3.83 (d, 1H), 3.30 (d, 1H), 3.21 (m, 1H), 3.03 (d, 1H), 2.71 (d, 1H), 2.42 (m, 8H), 2.29 (s, 3H), 2.04 (dd, 1H), 1.95 (dd, 1H), 1.78 (d, 1H); ATP-NMR (CDCl₃): δ 169.9 (s), 146.9 (s) 144.9 (s) 133.4 (s), 129.1 (d), 128.3 (s), 126.6 (d), 116.3 (d), 113.4 (s), 88.8 (d), 62.0 (d), 61.4 (t) 56.6 (q), 55.4 (2t), 53.7 (2t), 52.0 (s), 49.5 (t), 46.6 (q), 45.2 (t), 35.9 (t), 30.1 (t);

### EXAMPLE 8

### (4aR,6R,8aR)-11-(3-(4-methylpiperazine)-1-yl-propyl)-3-methoxy-5,6,9,10,11,12 -hexahydro-4aH-[1]benzofuro[3a,3,2-ef][2]benzazepin-6-ol, Trihydrochloride (Ib Y₁=OH, Y₂=H, X=Br, Z₁=C₇H₁₇N₂)

Step 1

2 g (+)-bromonorgalanthamine, prepared in accordance with WO-A-97/40049, and 5.6 mL 1-bromo-3-chloropropane and 3.92 g potassium carbonate are stirred in 10 mL acetonitrile at 80° C. for 4.5 h. After filtering out the potassium carbonate 70 mL water is added, the mixture is acidified with 2N HCl and extracted 2 times, each time with 30 mL ethyl acetate. The aqueous phase is made basic with 2N sodium hydroxide solution and extracted by shaking 2 times, each time with 50 mL dichloromethane. After separating the solvent, 1.12 g (46% of theory) yellowish oil remains. The product is immediately processed further.

Step 2

1.1 g N-(3-chloropropyl)-(+)-bromonorgalanthamine (

Step 1), 2.85 mL N-methyl piperazine and 2.1 g potassium carbonate are stirred for 3 h in 8 mL acetonitrile at 900C. The potassium carbonate is filtered out, and the solvent is concentrated. The resulting 2.27 g are chromatographed on 170 g silica gel using chloroform:methanol:ammonia solution=90:9:1 as eluent. The fractions containing product are concentrated, the residue is dissolved in 15 mL ether, and acidified at 0° C. with an ether solution of HCl. After filtering and washing two times, each time with 5 mL ether, the product is dried at 30° C. for 16 h in a vacuum dryer at 50 mbar.

Yield: 440 mg white crystals (32% of theory) M.p.: 220-238° C. R_{f}: 0.37 (chloroform:methanol: ammonia solution=90:9:1)

¹H-NMR (DMSO): δ 7.19 (s, 1H), 6.21 (d, 1H), 5.89 (d, 1H), 4.89 (d, 1H), 4.70 (b, 1H), 4.55 (d, 1H), 4.09 (b, 1H), 3.81 (s, 3H), 3.01-3.80 (m, 12H), 2.80 (m, 3H), 2.02 (s, 3H), 2.31 (m, 2H), 2.08 (m, 2H), 1.85 (b, 1H); ATP-NMR (DMSO): δ 172.7 (s), 147.3 (s) 146.3 (s) 134.8 (s), 132.1 (d), 131.9 (d), 125.7 (d), 117.1 (d), 115.0 (s), 87.4 (d), 65.7 (2t), 65.0 (2t), 60.1 (d), 53.2 (q), 49.9 (d), 48.2 (d), 43.7 (s), 42.8 (q), 41.1 (d), 41.0 (d), 40.9 (d), 31.4 (t);

### EXAMPLE 9

### Methyl-4-((4aR,6S,8aR)-1-bromo-4a,5,9,10,11,12-hexahydro-6-hydroxy-3-methoxy-6H-benzofuro[3a,3,2-ef][2]benzazepine-11-yl)gamma-oxo-butyrate (Ib Y₁=OH, Y₂=H, X=Br, Z₁=C₅H₇0₃)

Step 1:

2.0 g (+)-bromonorgalanthamine, prepared in accordance with WO-A-97/40049, 1.18 mL triethylamine and 0.6 g succinyl anhydride are vigorously stirred in 70 mL tetrahydrofuran at 75° C. After 30 min the reaction mixture is cooled, the solvents are vacuum distilled out and the residue is mixed with 100 mL 2N HCl and 50 mL ethyl acetate. After separating the organic phase, the aqueous phase is extracted with 2×50 mL ethyl acetate. The combined organic phases are dried over sodium sulfate, filtered, and vacuum concentrated.

Yield: 1.91 g yellowish foam

Step 2:

1.91 g foam, as prepared in Step 1, is dissolved in 20 mL methanol mixed with 0.6 mL dimethyl sulfate and stirred at room temperature (RT). After 24 h the reaction mixture is mixed with 50 mL water and 40 mL ethyl acetate. After separating the organic phase, the aqueous phase is extracted with 2×30 mL ethyl acetate. The combined organic phases are dried over sodium sulfate, filtered, and vacuum concentrated. The product is purified by column chromatography (ethyl acetate).

Yield: 0.54 g colorless oil (24.3% of theory) R_{f}: 0.35 (ethyl acetate)

¹H-NMR (DMSO): δ 7.25 (s, 1H), 6.12 (d, 1H), 5.81 (m, 1H), 5.01 (d, 1H), 4.69 (d, 1H), 4.51 (d,1H), 4.42 (m, 1H), 4.09 M, 1H), 3.78 (s, 3H), 3.53 (s, 3H), 3.29 (m, 1H), 2.77 (m, 1H), 2.52 (m, 3H), 2.28 (d, 1H), 2.04 (m, 1H), 1.85 (m, 1H), 1.69 (m, 1H); ATP-NMR (DMSO): δ 173.6 (s), 170.7 (s), 147.4 (s), 144.7 (s), 134.3 (s), 129.6 (d), 128.3 (s), 127.2 (d), 116.2 (d), 111.9 (s), 87.4 (d), 60.2 (d), 56.8 (q), 52.0 (q), 51.2 (t), 49.3 (s), 46.2 (t) 39.9 (t), 37.0 (t), 31.2 (t), 28.7 (t);

### EXAMPLE 10

### (4aR,6S,8aR)-11-(4-Aminopropyl)-3-methoxy-5,6,9,10,11,12-hexahydro-4aH-[1]benzofuro[3a,3,2-ef][2]benzazepine-6-ol, methane sulfonate (Ib Y₁=OH, Y₂=H, X=Br, Z₁=C₃H₈N)

Step 1:

2.0 g (+)-bromonorgalanthamine, prepared in accordance with WO-A-97/40049, and 7.2 mL 1-bromo-3-chloropropane and 5.0 g potassium carbonate are stirred in 10 mL acetonitrile at room temperature. After 19 h the precipitate is filtered out, and the filtrate is mixed 80 mL water, 25 mL 2N HCl and 50 mL ethyl acetate. After separating the organic phase the aqueous phase is extracted with 2×40 mL ethyl acetate. The combined organic phases are dried over sodium sulfate, filtered, and vacuum concentrated.

Yield: 1.5 g colorless foam.

Step 2:

1.5 g foam, as prepared in Step 1, is dissolved 15 mL methanol, mixed with 15 g NH4CI and 150 mL 25% ammonia solution and stirred at room temperature. After 18 h the reaction mixture is mixed with 400 mL water and 75 mL chloroform. After separating the organic phase the aqueous phase is extracted with 2×75 mL chloroform. The combined organic phases are dried over sodium sulfate, filtered, and vacuum concentrated. The residue is dissolved in 5 mL tetrahydrofuran and acidified with methane sulfonic acid to pH 1. The resulting precipitate is separated, washed with tetrahydrofuran and dried in a vacuum chamber.

Yield: 1.3 g (71% of theory) M.p.: 63-67° C. R_{f}: 0.15 (chloroform:MeOH:ammonia solution=0:18:2)

¹H-NMR (DMSO): δ 7.88 (b, 2H, NH₂), 6.85 (s, 1H), 6.19 (d, 1H), 5.89 (d, 1H), 4.70 (d, 1H), 4.60 (b, 1H), 4.48 (b, 1H), 4.18 (m, 1H), 3.80 (s, 3H), 3.70 (m, 1H), 3.59 (b, 2H), 3.42 (m, 2H), 2.88 (b, 2H), 2.52 (m, 1H), 1.91-2.34 (m, 4H); ATP-NMR (DMSO): δ 147.3 (s) 145.6 (s) 133.6 (s), 130.4 (d), 126.5 (s), 123.6 (d), 112.9 (d), 112.1 (s), 87.3 (d), 7.9 (2t), 60.4 (d), 56.5 (q), 49.8 (t), 40.9 (t), 40.8 (s), 37.3 (t) 31.7 (t), 25.9 (t);

### EXAMPLE 11

### (4aR,6S,8aR)-1-Bromo-4a,5,9, 1 0-tetrahydro-6-hydroxy-3-methoxy-6H-[ I ]benzofuro-[3a, 3,2-ef] [2]benzazepine-11(1 2H)-thiocarbonic acid methylamide (Ib Y,=OH, Y₂=H, X=Br, Z₁=C₂H4NS)

2.0 g (+)-bromonorgalanthamine, prepared in accordance with WO-A-97/40049, and 0.7 g methyl isothiocyanate are stirred in 50 mL toluene at reflux temperature. After 16 h the reaction mixture is cooled to room temperature, the solvent is vacuum distilled out, and the residue is mixed with 200 mL 2N HCl and 50 mL ethyl acetate. After separating the organic phase the aqueous phase is extracted with 2×50 mL ethyl acetate. The combined organic phases are dried over sodium sulfate, filtered, and vacuum concentrated. The product is purified by column chromatography (chloroform:MeOH=95:5).

Yield: 2.2 g (93% of theory) M.p.: 98-102° C. R_{f}: 0.7 (chloroform:MeOH:ammonia solution=90:9:1)

¹H-NMR (DMSO): δ 7.38 (b, 1H, NH), 6.97 (s, 1H), 6.07 (d, 1H), 5.80 (dd, 1H), 4.51 (b, 1H), 4.37 (m, 2H), 4.09 (d, 1H), 3.80 (m,lH), 3.72 (s, 3H), 3.31 (b, 1H), 2.81 (s, 3H), 2.28 (d, 1H), 2.02 (d, 1H), 1.85 (t, 1H), 1.61(d, 1H); ATP-NMR (DMSO): δ 182.0 (s), 147.5 (s) 144.7 (s) 134.0 (s), 129.4 (d), 128.6 (s), 127.6 (d), 116.4 (d), 113.2 (s), 87.3 (d), 60.2 (d), 56.8 (q), 57.1 (t), 49.1 (s), 48.4 (t) 36.7 (t), 33.7 (q), 31.2 (t);

### EXAMPLE 12

### (4aS,6R,8aS)-1-Bromo-6-hydroxy-3-methoxy-4a,5,9,10-tetrahydro-6H-[ 1]benzofuro[3a, 3,2-ef][2]benzazepine-11(12H)-thiocarbonic acid allylamide (Ia Y₁=OH, Y₂=H, X=Br, Z₁=C₄H₆NS)

2.0 g (−)-bromonorgalanthamine, prepared in accordance with WO-A-97/40049 and 0.6 mL allyl isothiocyanate are stirred in 60 mL tetrahydrofuran at reflux temperature. After 9 h the reaction mixture is cooled to room temperature, the solvent is vacuum distilled out, and the residue is purified by column chromatography (chloroform:MeOH=98:2).

Yield: 2.14 g (83.5% oftheory) M.p.: 175-179° C. R_{f}: 0.45 (chloroform:MeOH=9:1)

¹H-NMR (DMSO): δ 7.41 (b, 1H, NH), 6.95 (s, 1H), 6.06 (d, 1H), 5.76 (m, 2H), 5.08 (t, 2H), 4.42 (m, 3H), 4.09 (m, 2H), 3.83 (m,lH), 3.72 (s, 3H), 2.51 (d, 2H), 2.28 (d, 1H), 2.05 (d, 1H), 1.88 (t, 2H), 1.76(t, 1H); ATP-NMR (DMSO): 6181.3 (s), 147.5 (s) 144.8 (s), 136.1 (d), 134.0 (s), 129.4 (d), 128.2 (s), 127.5 (d), 116.3 (d), 116.3 (s), 113.1 (d), 87.3 (d), 60.2 (d), 56.8 (q), 49.0 (t), 48.8 (s), 41.0 (t) 40.9 (t), 36.7 (t), 31.1 (t);

### EXAMPLE 13

### (4aS,6S,8aS)-4a,5,9,10,11,12-Hexahydro-3-methoxy-6H-[ 1]benzofuro[3a,3,2-ef][2] benzazepine-6-ol (Ia Y₁=H, Y₂=OH, X=H, Z₁=H)

1 g (−)-norgalanthamine, prepared in accordance with W-A-00/174820, is dissolved in 80 mL 2% HC1 and stirred at reflux for 3 h. The reaction mixture is allowed to cool to room temperature, made basic with concentrated aqueous ammonia solution and extracted 3 times, each time with 30 mL chloroform. The combined organic phases are dried over sodium sulfate, filtered and concentrated. The resulting 1.06 g is purified by column chromatography on 80 g silica gel with eluent chloroform:methanol:ammonia solution=90:9:1 and the resulting fractions containing the product are concentrated.

Yield: 690 mg white powder (69% of theory) M.p.: 151-155° C. R_{f}: 0.29 (chloroform:methanol:ammonia solution=90:9:1)

¹H-NMR (CDCl₃): δ 6.65 (d, 1H), 6.49 (d, 1H), 6.02 (d, 1H), 5.65 (d, 1H), 4.48 (b, 1H), 4.25 (m, 1H), 3.85 (d,lH), 3.74 (d, 1H), 3.65 (s, 3H), 3.15 (dd, 1H), 3.03 (m, 1H), 2.43 (m, 1H), 1.79 (m, 2H), 1.63 (t, 1H); ATP-NMR (CDCl₃): δ 147.6 (s), 43.8 (s), 135.1 (s), 134.2 (s), 133.3 (d), 127.3 (d), 120.3 (d), 112.0 (d), 88.7 (d), 62.2.(d), 56.4 (q), 53.8 (t), 49.0 (s), 47.5 (t) 39.7 (t), 32.9 (t);

### EXAMPLE 14

### 4aS,6S,8aS)-6-Hydroxy-3-methoxy-4a,5,9, 1 0-tetrahydro-6H-[ I ]benzofuro [3a,3,2-ef][2]benzazepine-11(12H)-thiocarbonic acid methylamide (Ia Y₁=H, Y₂=OH, X=H, Z₁=C₄H₈NO)

2.32 g (−)-epinorgalanthamine (Ia Y₁=H, Y₂=OH, X=H, Z₁=H) and 0.9 mL isopropyl isocyanate are stirred in 150 mL toluene at reflux temperature. After 16 h the reaction mixture is cooled to room temperature, the solvent is distilled out, and the residue is mixed with 200 mL 2N HCl and 50 mL ethyl acetate. After separating the organic phase the aqueous phase is extracted with 2×50 mL ethyl acetate. The combined organic phases are dried over sodium sulfate, filtered, and vacuum concentrated. The product is purified by column chromatography (chloroform:MeOH=98:2).

Yield: 1.49 g (49.1% of theory) M.p.: 182-186° C. R_{f}: 0.5 (chloroform:MeOH:ammonia solution=90:9:1)

¹H-NMR (CDCl₃): δ 6.69 (b, 2H), 5.98 (d, 1H), 5.81 (d, 1H), 4.61 (m, 2H), 4.48 (d, 1H), 4.32 (d, (1H), 4.21 (d, 1H), 3.89 (m, 1H), 3.87 (s, 3H), 3.42 (t, 1H), 2.79 (d, 1H), 2.01 (dt, 1H), 1.79 (dd, 11H), 1.61(d, 1H), 1.11 (d, 3H), 0.98 (d, 3H); ¹³C-NMR (CDCl₃): δ 157.0 (s), 148.3 (s) 144.8 (s) 132.9 (d), 132.8 (s), 129.4 (d), 126.4 (d), 120.0 (d), 111.3 (d), 88.8 (d), 63.3 (d), 56.3 (q), 51.8 (t), 48.8 (s), 46.0 (t), 42.9 (d), 37.7 (t), 32.6 (t), 23.9 (q), 23.6 (q);

### EXAMPLE 15

### 4aS,6S,8aS)-4a,5,9,10,11,12-Hexahydro-11-(2-(morpholin-4-yl)-ethyl)-3-methoxy-6H-[1]benzofuro[3a,3,2-ef][2]benzazepine-6-ol (Ia Y₁=H, Y₂=OH, X=H, Z₁=C₆H₁₂NO)

1.55 g (−)-epinorgalanthamine (Ia Y₁=H, Y₂=OH, X=H, Z₁=H), 2.35 g potassium carbonate and 1.11 g N-(2-chloroethyl)-morpholine hydrochloride are stirred in 30 mL acetonitrile at reflux temperature. After 48 h the reaction mixture is cooled to room temperature, the solvent is vacuum distilled out, and the residue is mixed with 200 mL 2N HCl and 40 mL ethyl acetate. After separation the organic phase is discarded. The aqueous phase is made basic with ammonia solution and extracted with 3×40 mL ethyl acetate. The combined organic phases are dried over sodium sulfate, filtered, and vacuum concentrated. The product is purified by column chromatography (chloroform:MeOH=9:1).

Yield: 0.51 g white foam (23.3% of theory) R_{f}: 0.5 (chloroform:MeOH=9:1)

¹H-NMR (CDCl₃): δ 6.69 (d, 1H), 6.57 (d, 1H), 6.07 (d, 1H), 5.78 (d, 1H), 4.61 (m, 2H), 4.18 (d, 1H), 4.35 (s, 3H), 3.80 (d, 1H), 3.65 (m, 4H), 3.31 (t, 1H), 3.09 (d, 1H), 2.69 (d, 1H), 2.57 (m, 2H), 2.50 (m, 5H), 2.28 (b, 1H), 2.19 (t, 1H), 1.72 (t, 1H), 1.59 (d, 1H); ¹³C-NMR (CDCl₃): δ 146.7 (s) 143.9 (s) 133.0 (s), 131.8 (d), 129.4 (d), 126.4 (d), 121.5 (d), 110.9 (d), 88.4 (d), 66.8 (2t), 63.0 (d), 57.7 (d), 57.1 (q), 55.8 (t), 54.1 (2t), 52.1 (s), 48.3 (t), 47.9 (t), 33.5 (t), 32.4 (t);

### EXAMPLE 16

### 4aS,6S,8aS)-4a,5,9,10,11,12-Hexahydro-3-methoxy-11-(2-pyrimidinyl)-6H -[1]benzofuro[3a,3,2-ef][2]benzazepine-6-ol (Ia Y₁=H, Y₂=OH, X=H, Z₁=C₃H₃N₂)

2.0 g (−)-epinorgalanthamine (Ia Y₁=H, Y₂=OH, X=H, Z₁=H), 2.45 g NaHCO₃ and 0.88 g 2-chloropyrimidine are stirred in 120 mL ethanol at reflux temperature. After 44 h the reaction mixture is cooled to room temperature, the solvent is vacuum distilled out and the residue is mixed with 120 mL water and 200 mL ethyl acetate. After separating the organic phase the aqueous phase is extracted with 2×100 mL ethyl acetate. The combined organic phases are dried over sodium sulfate, filtered, and vacuum concentrated., The product is purified by column chromatography (chloroform:MeOH=98:2).

Yield: 1.24 g (48.2% of theory) M.p.: 223-226° C. R_{f}: 0.65 (chloroform:MeOH=9:1)

¹H-NMR (DMSO): δ 8.30 (d, 2H), 6.72 (d, 1H), 6.65 (d, 1H), 6.54 (t, 1H), 6.20 (d, 1H), 5.72 (d, 1H), 5.29 (d, 1H), 5.08 (d, 1H), 4.79 (d, 1H), 4.48 (m, 2H), 4.25 (m, 1H), 3.68 (s, 3H), 2.45 (m, 1H), 1.95 (t, 1H), 1.78 (d, 1H), 1.65 (t, 1H); ¹³C-NMR (DMSO): δ 161.1 (s), 158.8 (s), 147.9 (s), 144.1 (s) 133.6 (s), 133.5 (2d), 130.6 (d), 126.9 (d), 122.1 (d), 111.9 (d), 110.7 (d), 88.4 (d), 62.2 (d), 56.4 (q), 48.8 (t), 45.5 (s), 41.0 (t), 36.5 (t), 32.8 (t);

### EXAMPLE 17

### 4aS,6S,8aS)-4a,5,9,10,11,12-Hexahydro-3-methoxy-11-(2-methyl-prop-2-enyl)-6H -[1]benzofuro[3a,3,2-ef][2]benzazepine-6-ol (I Y₁=H, Y₂=OH, X=H, Z₁=C₄H₇)

2.0 g (−)-epinorgalanthamine (Ia Y₁=H, Y₂=OH, X=H, Z₁=H), 2.02 g potassium carbonate, 1.27 g potassium iodide and 0.85 mL 3-chloro-2-methyl-1-propene are stirred in 80 mL acetone at reflux temperature. After 48 h the reaction mixture is cooled to room temperature, the solvent is vacuum distilled out and the residue is mixed with 200 mL 2N HCl and 50 mL ethyl acetate. After separation, the organic phase is discarded. The aqueous phase is made basic with 30% sodium hydroxide solution and extracted with 3×100 mL ethyl acetate. The combined organic phases are dried over sodium sulfate, filtered, and vacuum concentrated. The product is purified by column chromatography (chloroform:MeOH=95:5).

Yield: 1.8 g resinous oil (75.1% of theory) R_{f}: 0.65 (chloroform:MeOH=95:5)

¹H-NMR (CDCl₃): δ 6.72 (d, 1H), 6.53 (d, 1H), 6.11 (d, 1H), 5.82 (d, 1H), 4.85 (d, 2H), 4.60 (m, 1H), 4.54 (b,1H), 4.09 (d, 1H), 3.87 (s, 3H), 3.67 (d, 1H), 3.32 (t, 1H), 3.05 (m, 2H), 2.83 (d, 1H), 2.18 (dt, 1H), 1.93 (b, 1H), 1.65 (s, 3H), 1.71 (d, 1H), 1.59 (d, 1H); ¹³C-NMR (CDCl₃): δ 146.7 (s) 143.9 (s) 133.0 (s), 131.8 (d), 129.4 (d), 126.4 (d), 121.5 (d), 110.9 (d), 88.4 (d), 66.8 (2t), 63.0 (d), 57.7 (d), 57.1 (q), 55.8 (t), 54.1 (2t), 52.1 (s), 48.3 (t), 47.9 (t), 33.5 (t), 32.4 (t);

### EXAMPLE 18

### 4aS,6S,8aS)-4a,5,9,10,11,12-Hexahydro-3-methoxy-11-propargyl-6H-[ I ]benzofuro[3a, 3,2-ef][2]benzazepine-6-ol (Ia Y₁=H, Y₂=OH, X=H, Z₁=C₃H₃)

2.6 g (−)-epinorgalanthamine (Ia Y₁=H, Y₂=OH, X=H, Z₁=H), 6.1 g potassium carbonate, 3.64 g potassium iodide and 1.47 mL 3-bromo-1-propyne are stirred in 150 mL acetonitrile at reflux temperature. After 12 h the reaction mixture is cooled to room temperature, the solvent is vacuum distilled out, and the residue is mixed with 300 mL 2N HCl and 100 mL ethyl acetate. After separation the organic phase is discarded. The aqueous phase is made basic with ammonia solution and extracted with 3×1O0 mL methylene chloride. The combined organic phases are dried over sodium sulfate, filtered and vacuum concentrated. The product is purified by column chromatography (chloroform:MeOH=95:5).

Yield: 0.8 g (26.1% of theory) M.p.: 157-160° C. R_{f}: 0.45 (chloroform:MeOH=95:5)

¹H-NMR (CDCl₃): δ 6.65 (d, 1H), 6.58 (d, 1H), 6.08 (d, 1H), 5.85 (d, 1H), 4.72 (m, 1H), 4.65 (b, 1H), 4.12 (d,lH), 3.87 (s, 3H), 3.79 (d, 1H), 3.38 (s, 2H), 3.31 (m, 1H), 3.20 (d, 1H), 2.45 (b, 1H), 2.31 (s, 1H), 2.10 (dt, 1H), 1.72 (m, 2H); ¹³C-NMR (CDCl₃): δ 146.7 (s) 143.9 (s) 132.9 (s), 131.9 (d), 128.5 (s), 126.4 (d), 121.6 (d), 111.0 (d), 88.4 (d), 79.5 (s), 72.9 (t), 63.0 (d), 58.0 (t), 55.9 (q), 51.7 (t), 48.0 (t), 43.9 (s), 34.9 (t), 32.3 (t);

### EXAMPLE 19

### 4aS,6S,8aS)-4a,5,9,10,11,12-Hexahydro-3-methoxy-11-benzoyl-6H-[ I ]benzofuro[3a,3,2 -ef][2]benzazepine-6-ol (Ia Y₁=H, Y₂=OH, X=H, Z₁=C₇H₅0)

2.0 g (−)-epinorgalanthamine (Ia Y₁=H, Y₂=OH, X=H, Z₁=H), 3.0 g potassium carbonate and 0.9 mL benzoyl chloride is stirred in 50 mL acetonitrile at reflux temperature. After 1 h, the reaction mixture is cooled to room temperature, the solvent is vacuum distilled out, and the residue is mixed with 100 mL water and 50 mL ethyl acetate. After separating the organic phase the aqueous phase is extracted with 2×50 mL ethyl acetate. The combined organic phases are washed with 2×40 mL 1N HCl and lx20 mL saturated NaCl solution (brine), dried over sodium sulfate, filtered, and vacuum concentrated. The residue is crystallized from 2-butanone.

Yield: 1.5 g (54% of theory) M.p.: 198-199° C. R_{f}: 0.4 (chloroform:MeOH:ammonia solution=95:4.5:0.5)

¹H-NMR (DMSO): δ 7.61 (m, 4H), 7.18 (d, 1H), 6.69 (m, 2H), 6.12 (d, 1H), 5.78 (b, 1H), 4.61 (b, 2H), 4.28 (b, 2H), 3.71 (s, 3H), 3.53 (m, 1H), 3.52 (m, 2H), 1.92 (m, 2H), 1.63 (m, 2H); ¹³C-NMR (DMSO): 8170.2 (s) 147.1 (s) 143. 5 (s), 136.6 (s), 132.9 (s), 132.7 (d), 128.7 (s), 128.0 (d), 126.2 (d), 126.0 (d), 125.8 (d), 125.5 (d), 120.8 (d), 119.3 (d), 111.3 (d), 87.4 (d), 61.1 (d), 55.4 (q), 53.1 (t), 48.1 (s), 47.3 (t), 43.1 (t), 31.8 (t);

### EXAMPLE 20

### 4aS,6S,8aS)-6-Hydroxy-3-methoxy-4a,5,9,10-tetrahydro-6H-[ l]benzofuro[3a,3,2-ef] [2] benzazepine-11(12H)-thiocarbonic acid allylamide (Ia Y₁=H, Y₂=OH, X=H, Z₁=c₇H₅0)

1.5 g (−)-epinorgalanthamine (Ia Y₁=H, Y₂=OH, X=H, Z₁=H) and 0.6 mL allyl isothiocyanate are stirred in 50 mL tetrahydrofuran at reflux temperature. After 3 h the reaction mixture is cooled to room temperature and the solvent is vacuum distilled out. The residue is purified by column chromatography (chloroform:MeOH=97:3).

Yield: 1.9 g white foam (92% of theory) R_{f}: 0.25 (chloroform:MeOH=97:3)

¹H-NMR (CDCl₃): δ 6.67 (m, 2H), 6.01 (d, 1H), 5.88 (m, 2H), 5.50 (b, 1H), 5.31 (d, 1H), 5.09 (t, 2H), 4.02 (d, 1H), 4.61 (m, 2H), 4.23 (m, 2H), 3.85 (s, 3H), 3.60 (t, 1H), 2.78 (d, 1H), 2.22 (t, 1H), 1.88 (d, 1H), 1.75 (t, 1H); ATP-NMR (DMSO): δ 181.6 (s), 148.7 (s) 145.3 (s) 134.2 (d), 132.8 (d), 126.8 (s), 126.4 (d), 1120.3 (d), 117.2 (s), 111.5 (d), 88.7 (d), 63.4 (d), 56.4 (q), 54.0 (t), 51.4 (s), 48.9 (t), 48.7 (t), 36.7 (t), 32.4 (t);

### EXAMPLE 21

### 4aS,6S,8aS)-4a,5,9,10-Tetrahydro-6-hydroxy-3-methoxy-6H-[ l ]benzofuro[3a,3,2-ef][2]benzazepine-11(12H)-carboxamide (Ia Y₁=H, Y₂=OH, X=H, Z₁=CH₂NO)

2.2 g (−)-epinorgalanthamine (Ia Y₁=H, Y₂=OH, X=H, Z,=H) and 1.05 g sodium cyanide are stirred in 112 mL water at room temperature and mixed in portions with 16 mL 2N HCl. After 24 h the precipitate is filtered out, washed with 2×2 mL water and dried in a vacuum dryer for 18 h at 50° C.

Yield: 0.56 g (22% of theory) M.p.: 168-173° C. R_{f}: 0.25 (chloroform:MeOH=9:1)

¹H-NMR (DMSO): δ 6.62 (d, 1H), 6.49 (d, 1H), 6.00 (d, 1H), 5.58 (d, 1H), 4.47 (b, 1H), 4.26 (t, 1H), 3.85 (d, 1H), 3.72 (d, 1H), 3.70 (s, 3H), 3.09 (m, 2H), 2.45 (m, 2H), 1.75 (m, 1H), 1.59 (t, (1H); ¹³C-NMR (DMSO): 6147.6 (s) 143.8 (s) 135.1 (s), 134.2 (s), 133.3 (d), 127.2 (d), 120.3 (d), 112.0 (d), 88.7 (d), 62.2 (d), 56.4 (q), 53.8 (t), 49.0 (s), 47.4 (t), 41.1 (t), 32.9 (t);

### EXAMPLE 22

### (4aS,6S,8aS)-4a,5,9,10,11,12-Hexahydro-3-methoxy-11-(3-methylbut-2-en-1-yl)-6H -[1ofuro[3a,3,2-ef][2]benzazepine-6-ol (Ia Y₁=H, Y₂=OH, X=H, Z₁=C₅H₉)

2.0 g (−)-epinorgalanthamine (Ia Y₁=H, Y₂=OH, X=H, Z₁=H), 2.02 g potassium carbonate and 1.0 mL 3,3-dimethylallyl bromide are stirred in 80 mL acetonitrile at reflux temperature. After 48 h the reaction mixture is cooled to room temperature, the solvent is vacuum distilled out, and the residue is mixed with 200 mL 2N HC1 and 50 mL ethyl acetate. After separation the organic phase is discarded. The aqueous phase is made basic with ammonia solution and extracted with 3×100 mL ethyl acetate. The combined organic phases are dried over sodium sulfate, filtered and vacuum concentrated. The product is purified by column chromatography (chloroform:MeOH=9:1).

Yield: 1.93 g (77% of theory) M.p.: 36-48° C. R_{f}: 0.2 (chloroform:MeOH:ammonia solution=90:9:1)

¹H-NMR (DMSO): δ 6.63 (d, 1H), 6.48 (d, 1H), 6.01 (d, 1H), 5.67 (d, 1H), 3.25 (m, 1H), 3.04 (d, 1H), 4.49 (s, 1H), 4.24 (b, 1H), 3.99 (d, 1H), 3.71 (s, 3H), 3.58 (d, 1H), 3.21 (t, 1H), 3.10 (m, 2H), 2.48 (m, 2H), 2.01 (dt, 1H), 1.68 (s, 3H), 1.62 (dt, 1H), 1.43 (s, 3H); ¹³C-NMR (DMSO): δ 147.1 (s), 144.0 (s) 134.4 (s), 133.5 (d), 130.6 (s), 126.7 (d), 123.2 (d), 121.1 (d), 112.1 (d), 88.7 (d), 62.2 (d), 57.8 (s), 56.3 (q), 51.8 (t), 50.4 (s), 48.6 (t), 41.0 (t), 40.0 (t), 39.7 (t) 26.6 (q), 18.8 (q);

### EXAMPLE 23

### (4aS,6S,8aS)-1-Bromo-11-(4-brombenzyl)-4a,5,9,10,11,12-hexahydro-3-methoxy-6H -[1]benzofuro[3a,3,2-ef][2]benzazepine-6-ol (Ia Y₁=H, Y₂=OH, X=H, Z₁=C₇H₄Br)

2.0 g (−)-epinorgalanthamine (Ia Y₁=H, Y₂=OH, X=H, Z₁=H), 6.0 g potassium carbonate and 1.92 g 4-bromobenzyl bromide is stirred in 40 mL tetrahydrofuran at room temperature. After 12 h the reaction mixture is cooled to room temperature, the precipitate is filtered out and vacuum concentrated. The product is purified by colurnn chromatography (chloroform:MeOH=95:5).

Yield: 1.81 g (56% oftheory) M.p.: 77-100° C. R_{f}: 0.3 (chloroform:MeOH:ammonia solution=90:9:1)

¹H-NMR (DMSO): δ 7.47 (d, 2H), 7.21 (d, 2H), 6.68 (d, 1H), 6.39 (d, 1H), 6.08 (d, 1H), 5.69 (d, 1H), 3.98 (d, 1H), 4.56 (b, 1H), 4.11 (d, 1H), 3.71 (s, 3H), 4.50 (m, 3H), 3.32 (m, 1H), 2.95 (d, 1H), 2.49 (m, 1H), 2.13 (t, 1H), 1.62 (t, 1H), 1.48 (d, 1H); ¹³C-NMR (DMSO): δ 147.3 (s), 144.2 (s), 139.6 (s) 134.2 (s), 133.6 (d), 131.9 (2d), 131.6 (2d), 130.2 (s), 126.6 (d), 121.8 (d), 120.6 (s), 112.2 (d), 88.6 (d), 62.2 (d), 57.6 (t), 55.3 (q), 51.6 (t), 48.7 (s), 41.0 (t), 34.0 (t), 33.0 (t);

### EXAMPLE 24

### (4aR,6R,8R)-4a,5,9,10,11,12-Hexahydro-3-methoxy-6H-[ 1]benzofuro[3a,3,2-efl [2]benzazepine-6-ol (Ib Y₁=H, Y₂=OH, X=H, Z,=H) 10 g (+)-norgalanthamine, prepared in accordance with WO-A-01/74820, is stirred in 400 mL 2% HCl solution under reflux cooling at the boiling point. After 3 h the reaction mixture is cooled, made basic with cc ammonia solution and the precipitate is separated.

Yield: 7.6 g (76% of theory) M.p.: 166-168° C. R_{f}: 0.2 (chloroform:MeOH:ammonia solution=90:9:1)

¹H-NMR (CDCl₃): δ 6.70 (d, 1H), 6.62 (d, 1H), 6.00 (d, 1H), 5.78 (d, 1H), 4.68 (b, 2H), 3.95 (d, 1H), 3.85 (d, 1H), 3.79 (s, 3H), 3.32 (d, 1H), 3.20 (t, 1H), 2.75 (m, 1H), 1.90 (d, 1H), 1.82 (dt, 1H), 1.59 (dt, 1H); ¹³C-NMR (CDCl₃): δ 147.5 (s) 144.2 (s) 133.5 (s), 133.4 (s), 132.5 (d), 126.9 (d), 120.5 (d), 111.2 (d), 88.9 (d), 63.0 (d), 56.3 (q), 54.1 (t), 49.0 (s), 47.6 (t), 41.4 (t), 32.7 (t);

### EXAMPLE 25

### (4aR,6R,8aR)-4a,5,9,10-Tetrahydro-6-hydroxy-3-methoxy-6H-[ 1]benzofuro[3a,3,2-ef] [2]benzazepine-11(12H)-carboxamide (Ib Y₁=H, Y₂=OH, X=H, Z₁=CH₂NO)

2.2 g (+)-epinorgalanthamine (Ib Y₁=H, Y₂=OH, X=H, Z=H) is dissolved in 112 mL twice distilled water, adjusted to pH=3 with 2N HCl, 1.05 g NaOCN is added, and the pH is again adjusted to 3 with 2N HCl. The reaction mixture is stirred for 20 h at room temperature; the resulting precipitate is filtered out and dried for 20 h in a vacuum chamber at 50 mbar and 60° C. The 2.2 g of crude product that is obtained is dissolved in 15 mL MeOH while heating at reflux, stirred for 1 h on an ice bath, and filtered.

Yield: 1.1 g white crystals (43.2% of theory) FP: 208-214° C. Rf.: 0.45 (chloroform:MeOH:ammonia solution=90:9:1)

¹H-NMR (DMSO): δ 6.72 (d, 1H), 6.68 (d, 1H), 6.08 (d, 1H), 5.87 (b, 2H, NH2), 5.69 (d, 1H), 5.00 (d, 1H), 4.59 (d, 1H), 4.48 (s, 1H), 4.28 (m, 1H), 4.11 (m, 1H), 3.71 (s, 3H), 3.38 (m, 2H), 2.49 (m, 2H), 1.88 (dt, 1H), 1.62 (m, 2H); ¹³C-NMR (DMSO): δ 158.3 (s), 147.8 (s), 144.2 (s) 133.6 (d), 133.5 (s), 131.1 (s), 126.8 (d), 121.3 (d), 112.0 (d), 88.5 (d), 62.2 (d), 56.4 (q), 48.7 (t), 45.5 (s), 41.0 (t), 37.9 (t), 32.8 (t);

### EXAMPLE 26

### (4aR,6R,8aR)-4a,5,9,10,11,12-Hexahydro-3-methoxy-1-ethyl-6H-[ I ]benzofuro[3a,3,2 -ef][2]benzazepine-6-ol (Ib Y₁=H, Y₂=OH, X=H, Z₁=C₂H₅)

1.35 g (+)-epinorgalanthamine (Ib Y₁=H, Y₂=OH, X=H, Z₁=H), 2.0 g potassium carbonate and 0.8 mL ethyl bromide is stirred in 50 mL tetrahydrofuran at reflux temperature. After 70 h the reaction mixture is cooled to room temperature, the solvent is vacuum distilled out, and the residue is mixed with 50 mL water and 50 mL ethyl acetate. After separating the organic phase the aqueous phase is extracted with 2×50 mL ethyl acetate. The combined organic phases are dried over sodium sulfate, filtered and vacuum concentrated. The product is purified by column chromatography (chloroform:MeOH:ammonia solution=95:4.5:0.5).

Yield: 1.0 g (67.2% of theory) M.p.: 135-136° C. R_{f}: 0.2 (chloroform:MeOH:ammonia solution=95:4.5:0.5)

¹H-NMR (DMSO): δ 6.63 (d, 1H), 6.50 (d, 1H), 6.05 (d, 1H), 5.68 (d, 1H), 4.95 (b, 1H), 4.48 (s, 1H), 4.27 (b, 1H), 4.02 (d, 1H), 3.72 (s, 3H), 3.68 (d, 1H), 3.29 (t, 1H), 3.08 (d, 1H), 2.41 (m, 2H), 2.03 (t, 1H), 1.62 (t, 1H), 1.57 (d, 1H), 0.91 (t, 3H); ¹³C-NMR (DMSO): δ 147.2 (s) 144.0 (s) 134.1 (s), 133.5 (d), 130.3 (s), 126.7 (d), 121.8 (d), 112.1 (d), 88.6 (d), 62.2 (d), 56.9 (d), 56.3 (q), 51.7 (t), 48.7 (s), 45.5 (t), 33.7 (t), 33.0 (t), 13.5 (q);

### EXAMPLE 27

### Methyl(4aR,6R,8aR)-N 1l -cyano-6-hydroxy-3-methoxy-4a,5,9,10-tetrahydro-6H -[1 ]benzofuro[3a,3,2-ef] [2]benzazepine-11(12H)-carboximidothioate (Ib Y=H, Y₂=OH, X=H, Z₁=C₂H₅)

2.5 g (+)-epinorgalanthamine (Ib Y₁=H, Y₂=OH, X=H, Z₁=H) and 1.05 g dimethyl-N-cyanodithioiminocarbonate is stirred in 80 mL ethanol and 20 mL dimethyl formamide at reflux temperature. After 21 h the reaction mixture is cooled to room temperature and the solvent is vacuum distilled out. The residue is purified by column chromatography (chloroform:MeOH=98:2).

Yield: 0.72 g (20.5% of theory) M.p.: 78-79° C. R_{f}: 0.35 (chloroform:MeOH:ammonia solution=90:9:1)

¹H-NMR (DMSO): o 6.72 (m, 2H), 6.12 (d, 1H), 5.72 (d, 1H), 5.05 (m, 2H), 6.19 (m, 2H), 4.26 (b, 1H), 3.72 (s, 3H), 3.32 (b, 1H), 2.62 (s, 3H), 2.49 (m, 1H), 1.89 (m, 2H), 1.65 (m, 1H); ¹³C-NMR (DMSO): δ 148.0 (s), 144.9 (s) 134.1 (d), 133.0 (s), 127.3 (s), 126.3 (d), 121.7 (d), 115.5 (s), 115.0 (s), 112.3 (d), 88.2 (d), 62.0 (d), 56.4 (q), 48.4 (t), 41.0 (s), 40.9 (t), 40.8 (t), 32.7 (t), 16.6(q);

### EXAMPLE 28

### (4aR,6R,8aR)-4a,5,9,10,11,12-Hexahydro-3-methoxy-11-methyl-6H-benzofuro[3a,3,2 -ef][2]benzazepine-6-amine, dihydrochloride

3.0 g (+)-galanthamine, prepared in accordance with Kametani, Heterocycles 4, 1111, 1976, and 3.3 g triphenyl phosphine and 18 mL hydroazoic acid (1.06 mol/L in benzene) are dissolved in 225 mL tetrahydrofuran, mixed with 7.0 mL azodicarboxylic acid diethyl ester (40% in toluene) at room temperature and vigorously stirred. After 20 h the reaction mixture is mixed with 150 mL 2N HCl, vigorously stirred for 1 h, the organic phase is separated, and the aqueous phase is washed with 2×50 mL ethyl acetate. The pH of the aqueous phase is adjusted to 12 with an ammonia solution and the cloudy suspension is extracted with 3×80 mL methylene chloride. The combined organic phases are dried over sodium sulfate, filtered, and vacuum concentrated. The product is purified by column chromatography (chloroform:MeOH:ammonia solution=95:4.5:0.5). The resulting oil is dissolved in isopropanol and the hydrochloride salt precipitates as etheric HCl.

Yield: 1.54 g (41% of theory) M.p.: 235-250° C. R_{f}: 0.45 free base (chloroformn:MeOH:ammonia solution=95:4.5:0.5)

¹H-NMR (CDCl₃): 8 6.69 (d, 1H), 6.60 (d, 1H), 6.21 (d, 1H), 5.75 (d, 1H), 4.62 (b, 1H), 4.37 (m, 1H), 4.08 (d, 1H), 3.89 (s, 3H), 3.67 (d, 1H), 3.29 (t, 1H), 3.09 (d, 1H), 2.81 (m, 1H), 2.41 (s, 3H), 2.22 (dt, 1H), 1.85 (dt, 1H), 1.67 (d, 1H); ¹³C-NMR (CDCl₃): δ 146.9 (s) 144.3 (s) 132.8 (s), 129.8 (s), 129.3 (d), 126.9 (d), 122.1 (d), 111.5 (d), 87.9 (d), 60.8 (d), 56.3 (q), 54.4 (t), 53.8 (q), 48.6 (s), 42.5 (d), 34.7 (t), 29.1 (t);

### EXAMPLE 29

### (4aR,6S,8aR)-11-(3-Hydroxypropyl)-3-methoxy-5,6,9,10,11,12-hexahydro-4aH-[1 ]benzo furo[3a,3,2-ef][2]benzazepine-6-ol (Ib Y₁=OH, Y₂=H, X=H, Z₁=C₃H₇0)

1.3 g (+)-norgalanthamine, prepared in accordance with WO-A-01/74820, and 2.6 g potassium carbonate and 0.65 mL 3-bromo-1-propanol are stirred in 70 mL acetonitrile at reflux temperature. After 48 h the reaction mixture is cooled to room temperature, the solvent is vacuum distilled out and the residue is mixed with 90 mL water and stirred in 40 mL chloroform. After separating the organic phase the aqueous phase is extracted with 2×30 mL chloroform. The combined organic phases are dried over sodium sulfate, filtered and vacuum concentrated. The product is purified by column chromatography (chloroform:MeOH=9:1).

Yield: 0.86 g resinous oil (54.5% of theory) R_{f}: 0.45 (chloroform:MeOH=9:1)

¹H-NMR (CDCl₃): δ 6.66 (m, 2H), 6.05 (m, 2H), 4.59 (b, 1H), 4.14 (m, 1H), 4.09 (d, 1H), 3.95 (d, 1H), 3.81 (s, 3H), 3.79 (t, 2H), 3.31 (m, 2H), 2.75 (m, 3H), 2.05 (m, 2H), 1.78 (m, 1H), 1.59 (m, 2H); ¹³C-NMR (CDCl₃): δ 146.2 (s) 144.7 (s) 133.5 (s), 128.7 (s), 128.2 (d), 127.0 (d), 122.7 (d), 111.6 (d), 89.1 (d), 64.8 (t), 62.4 (d), 57.8 (d), 56.3 (q), 52.4 (t), 52.1 (t), 48.7 (s), 33.3 (t), 30.3 (t), 27.9 (t);

### EXAMPLE 30

### (4aS,6R,8aS)-6-Hydroxy-3-methoxy-5,6,9,10-tetrahydro-4aH-[1 ]benzofuro[3a,3,2-ef] [2]benzazepine-11(12H)-carbothioamide (Ib Y₁=OH, Y₂=H, X=H, Z₁I=CH₂NO)

2.2 g (+)-norgalanthamine, prepared in accordance with WO-A-01/74820, is dissolved in 112 mL twice distilled water, adjusted to pH=3 with 2N HCl, 1.05 g NaOCN is added and the pH is again adjusted to 3 with 2N HCl. The reaction mixture is stirred for 20 h at room temperature, made basic with a concentrated ammonia solution, and extracted by shaking 3 times, each time with 40 mL dichloromethane. The combined organic phases are dried over sodium sulfate, filtered, and vacuum concentrated. The resulting 2.5 g is purified by column chromatography on 170 g silica gel using the eluent chloroform:methanol=95:5, and the purified fractions are concentrated.

Yield: 1.08 g white crystals (42.4% of theory) M.p.: 101-114° C. R_{f}: 0.29 (chloroform:MeOH=95:5)

¹H-NMR (DMSO): δ 6.75 (d, 1H), 6.69 (d, 1H), 6.08 (d, 1H), 5.87 (b, 2H, NH2), 5.78 (dd, 1H), 5.60 (d, 1H), 4.54 (b, 1H), 4.22 (m, 2H), 4.05 (m, 1H), 3.70 (s, 3H), 3.39 (m, 1H), 2.29 (d, 1H), 2.05 (dd, 1H), 1.78 (t, 1H), 1.60 (d, 1H); ¹³C-NMR (DMSO): δ 158.3 (s), 147.6 (s), 144.2 (s) 133.1 (d), 131.1 (s), 129.0 (d), 127.8 (d), 121.1 (d), 111.9 (d), 87.3 (d), 60.6 (d), 56.4 (q), 51.0 (t), 48.6 (s), 45.5 (t), 38.1 (t), 31.6 (t);

### EXAMPLE 31

### (4aS,6S,8aS)-4a,5,9,10,11,12-Hexahydro-3-methoxy-11-benzoyl-6H-[ I ]benzofuro[3a,3,2 -ef] [2]benzazepine-6-ol

Step 1

A solution of 6.0 g (+)-galanthamine, prepared in accordance with Kametani, Heterocycles 4, 1111, 1976, in 42 mL glacial acetic acid is mixed with a mixture of 12.7 mL nitric acid and 21 mL glacial acetic acid at 0-5° C. After 15 min the reaction mixture is added by drops to 100 mL water, the pH is adjusted to 12 with an ammonia solution, and the mixture is mixed with 100 mL chloroform. After separating the organic phase the aqueous phase is extracted with 3×50 mL chloroform. The combined organic phases are dried over sodium sulfate, filtered and vacuum concentrated. The product is purified by column chromatography (chloroform:MeOH=95:5).

Yield: 1.47 g brown oil (19.4% of theory) R_{f}: 0.25 (chloroform:MeOH=95:5)

Step 2

1.47 g of the product obtained in Step 1, 2.94 g zinc powder and 1.47 g CaCl₂ are mixed in 44 mL ethanol and 22 mL water at reflux temperature. After 3 h the resulting precipitate is filtered out and the solvent is distilled out. The product is purified by column chromatography (chloroform:MeOH:ammonia solution=90:9:1).

Yield: 0.36 g (43.4% of theory) M.p.: 166-167° C. R_{f}: 0.3 (chloroformn:MeOH:ammonia solution=90:9:1)

¹H-NMR (DMSO): δ 6.18 (s, 1H), 6.04 (d, 1H), 5.78 (dd, 1H), 4.41 (b, 2H, NH2), 4.33 (b, 1H), 4.01 (d, 1H), 3.82 (d, 1H), 3.58 (s, 3H), 3.55 (d, 1H), 3.18 (t, 1H), 2.87 (d, 1H), 2.28 (s, 3H), 2.22 (d, 1H), 1.98 (d, 1H), 1.89 (t, 1H), 155 (d, 1H); ¹³C-NMR (DMSO): δ 158.3 (s), 147.6 (s), 144.2 (s) 133.1 (d), 131.1 (s), 129.0 (d), 127.8 (d), 121.1 (d), 111.9 (d), 87.3 (d), 60.6 (d), 56.4 (q), 51.0 (t), 48.6 (s), 45.5 (t), 38.1 (t), 31.6 (t);

### EXAMPLE 32

### 2-((4aS,6R,8aS)-6-Hydroxy-3-methoxy-5,6,9, 10-tetrahydro-4aH-[ 1 ]benzofuro[3a,3,2-ef] [2]benzazepine-11(12H)-yl)-1-methyl-i -(3-phenoxyphenyl)ethane hydrochloride (Ia Y₁=OH, Y₂=H, X=H, Z₁=Cl₅HI₅0)

Step 1

3.63 g (+/−)-2-[3-phenoxyphenyl]-1-propanoic acid is dissolved in 20 mL tetrahydrofuran, 700 mg lithium aluminium hydride is added and the mixture is stirred for 1 h at room temperature. Then 50 mL water is carefully added by drops, and the mixture is stirred for 1 h and filtered. The clear filtrate is extracted 3 times, each time with 20 mL ethyl acetate, the combined organic phases are dried with sodium sulfate, filtered and the solvent is removed.

Yield: 2.4 g colorless oil (70% of theory) R_{f}. 0.36 (petroleum ether:ethyl acetate=4:1)

Step 2

3.15 g triphenylphosphine is dissolved in 90 mL tetrahydrofuran, 0.6 mL bromine is added by drops, and 2.4 g (+/−)-2-[3-phenoxyphenyl]-1-propanol from Step 1 in solid state is added to the resulting suspension. After 30 min 100 mL water is added, the mixture is extracted 3 times, each time with 30 mL ethyl acetate, the organic phases are combined, dried over sodium sulfate, filtered and the clear filtrate is suctioned through a short silica gel column. After removing the solvent by evaporation, 2.7 g (88% of theory) of a colorless oil is obtained. The resulting (+/−)-2-[3-phenoxyphenyl]-1-bromopropane was immediately used for the next step.

Step 3

4.56 g (−)-norgalanthamine HCl, prepared in accordance with WO-A-01/74820, and 4 g 3-(1-bromo-2-propyl)diphenyl ether and 9.97 g potassium carbonate are stirred for 40 h in 53 mL acetonitrile at 85° C. The suspension is poured into 10 mL water and extracted 3 times, each time with 30 mL ethyl acetate. After drying the organic phase over sodium sulfate it is filtered and concentrated and the resulting 4.5 g is purified by column chromatography on 400 g silica gel with ethyl acetate. Solvent is removed from the product-containing fractions and they are taken up in 50 mL diethyl ether, and the hydrochloride is precipitated with etheric HCl.

Yield: 1.5 g (19% of theory) M.p.: 109-115° C. Rf.: 0.67 (ethyl acetate)

¹H-NMR (CDCl₃): δ 7.35 (m, 2H), 7.24 (m, 1H), 7.13 (m, 1H), 7.00 (m, 2H), 6.89 (m, 3H), 6.62(m, 2H), 6.04 (m, 2H), 4.60 (b, 1H), 4.14 (m, 2H), 3.85 (s, 3H), 3.77 (t, 1H), 3.38 (m, 1H), 3.12 (m, 1H), 2.91 (m, 1H), 2.70 (m, 2H), 2.48 (m, 1H), 1.99 (m, 2H), 1.48 (m, 1H), 1.25 (m, 3H); ¹³C-NMR (CDCl₃): δ 157.3 (s), 157.2 (s), 157.0 (s), 148.3 (s), 148.2 (s), 145.8 (s) 144.0 (s) 133.2 (s), 133.1 (s), 129.9 (s), 129.6 (d), 129.5 (d), 129.4 (d), 127.5 (d), 127.4 (d), 127.0 (d), 123.0 (d), 122.9 (d), 122.2 (d), 122.1 (d), 121.9 (d), 121.8 (d), 118.7 (d), 118.6 (d), 111.0 (d), 88.7 (d), 62.1 (d), 58.0(t), 57.0(t), 55.9 (q), 52.3 (t), 51.2 (t), 48.5 (s), 48.4 (s), 37.9 (d), 37.7 (d), 32.8 (t), 32.5 (t), 29.9 (t), 20.0 (q), 19.5(q);

### EXAMPLE 33

### (4aS,6R,8aS)-4a,5,9,10,11,12-Hexahydro-3-methoxy-11-benzoyl-6H-[1 ]benzofuro[3a,3, 2-ef][2]benzazepine-6-ol (Ia Y₁=OH, Y₂=H, X=H, Z₁=C₇H₅0).

2.0 g (−)-norgalanthamine, prepared in accordance with WO-A-01/74820, is dissolved in 50 mL acetonitrile with slight heating then cooled to 0-10° C. The reaction mixture is mixed with 3.0 g potassium carbonate and 0.9 mL benzoyl chloride and stirring is continued at room temperature. After 1 h, the solvent is vacuum distilled out, and the residue is mixed with 50 mL water and 20 mL ethyl acetate. After separating the organic phase the aqueous phase is extracted with 3×20 mL ethyl acetate. The combined organic phases are washed with 2×20 mL 1N HCl and lx20 mL water, dried over sodium sulfate, filtered and vacuum concentrated. The residue was crystallized from 2-butanone and tert-butyl methyl ether (MTBE).

Yield: 1.76 g (63.7% of theory) M.p.: 152-154° C. R_{f}: 0.75 (chloroform:MeOH:ammonia solution=95:4,5:0,5)

¹H-NMR (DMSO): δ 7.39 (m, 4H), 7.11 (d, 1H), 6.72 (b, 1H), 6.63 (d, 1H), 6.09 (m, 1H), 5.81 (b, 1H), 4.62 (d, 1H), 4.55 (b, 1H), 4.31 (m, 2H), 4.09 (d, 1H), 3.71 (s, 3H), 3.48 (m, 1H), 2.32 (t, 1H), 2.09 (m, 1H), 1.89 (b, 1H), 1.70 (m, 1H); ¹³C-NMR (DMSO): 6171.0 (s) 147.8 (s) 144.5 (s), 137.5 (s), 133.3 (s), 130.1 (d), 129.7 (s), 129.4 (d), 129.3 (d), 129.0 (d), 127.7 (d). 127.5 (d), 121.6 (d), 120.3 (d), 112.3 (d), 87.3 (d), 60.5 (d), 56.4 (q), 54.2 (t), 48.5 (s), 44.0 (t), 37.4 (t), 31.4 (t);

### EXAMPLE 34

### 2-((4aS,6R,8aS)-6-Hydroxy-3-methoxy-5,6,9,10-tetrahydro-4aH-[ 1]benzofuro[3a,3,2-efl [2]Benzazepine-11(12H)-yl)acetamide (Ia Y₁=OH, Y₂=H, X=H, Z₁=C₂H4NO)

3.0 g (−)-norgalanthamine HCl, prepared in accordance with WO-A-01/74820, and 3.0 g potassium carbonate and 1.4 g 2-bromacetamide are stirred in 50 mL acetonitrile at reflux. After 3 h the resulting precipitate is filtered while warm, the solvent is removed in a vacuum and the residue is recrystallized from ethanol.

Yield: 1.74 g (54.4% of theory) M.p.: 107-113° C. . R_{f}: 0.5 (chloroform:MeOH:ammonia solution=89:10:1)

¹H-NMR (DMSO): δ 7.18 (d, 2H, NH2), 6.71 (d, 1H), 6.52 (d, 1H), 6.07 (d, 1H), 5.81 (dd, 1H), 4.48 (b, 1H), 4.27 (d, 1H), 4.19 (d, 1H), 4.05 (b, 1H), 3.71 (s, 3H), 3.63 (d, 1H), 3.28 (t, 1H), 3.00 (d, 1H), 2.88 (d, 1H), 2.27 (d, 1H), 2.06 (m, 1H), 1.93 (t, 1H), 1.46 (d, 1H); ¹³C-NMR (DMSO): δ 173.2 (s), 147.1 (s), 144.3 (s) 133.6 (s), 130.2 (s), 129.2 (d), 127.7 (d), 121.9 (d), 112.1 (d), 87.6 (d), 60.8 (d), 58.5 (t), 56.4 (q), 56.3 (t), 52.6 (s), 48.6 (t), 35.1 (t), 31.7 (t);

### EXAMPLE 35

### 3-[(4aS,6R,8aS)-6-Hydroxy-3-methoxy-5,6,9,10-tetrahydro-4aH-[l ]benzofuro[3a,3,2-ef] [2]benzazepine-11 (1 2H)-yl]-2-methyl-1 (-4-methylphenyl)propan-1-one, hydrochloride (Ia Y₁=OH, Y₂=H, X=H, Z₁=C₁ IH_{1 3}0) 6.0 g (−)-norgalanthamine HCl, prepared in accordance with WO-A-01/74820, and 3.3 g 4-methylpropiophenone, 6.0 mL 1,3-dioxolane and 0.2 mL 2N HCl are heated under reflux. After 3 h the solvent is distilled out and the residue is mixed with 100 mL water and 50 mL ethyl acetate. The pH of the aqueous phase is adjusted to 9 with an ammonia solution. After separating the organic phase the aqueous phase is extracted with 3×50 mL ethyl acetate. The combined organic phases are washed with lx20 mL saturated NaCl solution (brine) and Ix20 mL water, dried over sodium sulfate, filtered and vacuum concentrated, and the product is purified by column chromatography (ethyl acetate:n-hexane=1:1 to 8:2). The resulting substance is taken up in ethyl acetate and hydrochloride salts are precipitated with etheric HCl.

Yield: 4.4 g (48% of theory) M.p.: 143-151° C. R_{f}: 0.4 (chloroform:MeOH=97:3)

¹H-NMR (CDCl₃): δ 7.84 (m, 2H), 7.26 (m, 2H), 6.68 (m, 2H), 6.05 (m, 2H), 4.61 (b, 1H), 4.14 (m, 2H), 3.86 (s, 3H), 3.71 (m, 2H), 3.34 (m, 1H), 3.12 (m, 2H), 2.69 (d, 1H), 2.55 (m, 1H), 2.30 (m, 4H), 2.03 (m, 2H), 1.49 (d, 1H), 1.15 (m, 3H); ¹³C-NMR (CDCI₃): δ 203.4 (s), 145.9 (s), 144.2 (s) 143.8 (s), 134.4 (s), 133.3 (s), 129.7 (s), 129.3 (d), 129.2 (d), 128.4 (d), 128.3 (d), 127.6 (d), 126.9 (d), 122.0 (d), 111.1 (d), 88.7 (d), 62.1 (d), 57.8 (t), 55.9 (q), 54.1 (t), 52.2 (t), 48.5 (s), 39.1 (d), 32.9 (t), 29.9 (t), 21.6 (q), 16.5 (q);

### EXAMPLE 36

### 1-[(4aS,6R,8aS)-6-Hydroxy-3-methoxy-5,6,9,10-tetrahydro-4aH-[ 1 ]benzofuro[3a,3,2-ef] [2]benzazepine-11(12H)-yl]-2-(1-piperidyl)ethan-1-one (Ia Y₁=OH, Y₂=H, X=H, Z₁=C₇H₁₂NO) 3.0 g (−)-norgalanthamine, prepared in accordance with WO-A-017/4820, and 1.86 mL triethylamine and 0.6 mL chroroacetylchloride are stirred in 150 mL tetrahydrofuran at 0° C. After 10 min the reaction mixture is mixed with 3.0 g potassium carbonate and 0.93 mL piperidine and stirring is continued at 90° C. After 48 h the solvent is distilled out, the residue is mixed with 50 mL water and 50 mL chloroform. After separating the organic phase the aqueous phase is extracted with 2×30 mL chloroform. The combined organic phases are dried over sodium sulfate, filtered and vacuum concentrated. The product is purified by column chromatography (chloroform:MeOH=95:5).

Yield: 2.06 g resinous oil (47.1% of theory) R_{f}: 0.3 (chloroform:MeOH=9:1)

¹H-NMR (CDCl₃): δ 6.69 (m, 2H), 5.98 (m, 2H), 5.19 (d, 1H), 4.56 (b, 1H), 4.37 (d, 1H), 4.15 (d,lH), 3.80 (s, 3H), 3.27 (d, 1H), 3.18 (m, 1H), 2.89 (d, 1H), 2.69 (d, 1H), 2.41 (m, 5H), 2.06 (d, 1H), 1.91 (m, 1H), 1.75 (d, 1H), 1.50 (m, 4H), 1.39 (b, 1H); ¹³C-NMR (CDCl₃): δ 196.5 (s), 146.8 (s), 144.6 (s), 132.5 (s), 128.7 (s), 128.0 (s), 28.2 (d), 126.5 (d), 122.0 (d), 111.3 (d), 88.4 (d), 62.3 (t), 61.8 (d), 55.9 (d), 55.8 (q), 55.6 (t), 52.2 (s), 45.0 (t), 38.6 (t), 35.7 (t), 29.9 (t), 25.8 (t), 23.9 (t);

### EXAMPLE 37

### (4aS,6R,8aS)-3-Methoxy-11-(2-pyrimidinyl)-5,6,9,10,11,12-hexahydro-4aH-[ 1 ]benzofur o[3a,3,2-ef][2]benzazepine-6-ol (Ia Y₁=OH, Y₂=H, X=H, Z₁=C₄H₃N₂)

2.0 g (−)-norgalanthamine, prepared in accordance with WO-A-01/74820, and 2.45 g NaHCO₃ and 0.88 mL 2-chloropyrimidine are stirred in 120 mL ethanol at the boiling point. After 44 h the solvent is distilled out, and the residue is mixed with 120 mL water and 100 mL ethyl acetate. After separating the organic phase the aqueous phase is extracted with 2xlOO mL ethyl acetate. The combined organic phases are dried over sodium sulfate, filtered, and vacuum concentrated. The product is purified by column chromatography (chloroform:MeOH=98:2).

Yield: 1.26 g (49% of theory) M.p.: 232-235° C. R_{f}: 0.7 (chloroform:MeOH:ammonia solution=89:10:1)

¹H-NMR (DMSO): δ 8.28 (m, 2H), 6.78 (d, 1H), 6.69 (d, 1H), 6.58 (t, 1H), 6.23 (d, 1H), 5.82 (dd, 1H), 5.25(d, 1H), 4.66 (d, 1H), 4.52 (d, 1H), 4.30 (b, 1H), 4.11 (b, 1H), 3.80 (s, 3H), 3.73 (m, 1H), 2.28 (d, 1H), 2.02 (d, 1H), 1.81 (t, 1H), 1.72 (d, 1H); ¹³C-NMR (DMSO): δ 161.2 (s), 158.7 (d), 147.6 (s), 144.1 (s) 133.3 (s), 130.6 (s), 129.0 (2d), 128.0 (d), 121.9 (d), 112.0 (d), 110.7 (d), 87.2 (d), 60.6 (d), 56.4 (q), 51.3 (t), 48.7 (s), 45.5 (t), 36.6 (t), 31.5 (t);

### EXAMPLE 38

### 1-[(4aS,6R,8aS)-6-Hydroxy-3-methoxy-5,6,9,10-tetrahydro-4aH-[ l ]benzoftiro[3a,3,2-ef] [2]benzazepine-11(12H)-yl]-3-(1-pyrrolidyl)propan-1-one (Ia Y₁=OH, Y₂=H, X=H, Z₁=C₇H₁₂NO)

2.0 g (−)-norgalanthamine, prepared in accordance with WO-A-017/4820, and 0.86 mL triethylamine and 0.76 mL 3-bromopropionyl chloride are stirred in 130 mL acetone at room temperature. After 60 min the solvent is distilled out and the residue is mixed with 100 mL 2N HCl solution and 50 mL ethyl acetate. After separating the organic phase the aqueous mother solution is extracted with 2×50 mL ethyl acetate. The combined organic phases are dried over sodium sulfate, filtered and vacuum concentrated. The oily residue is taken up in 50 mL acetonitrile, mixed with 5 mL pyrrolidine and stirred at the boiling point. After 8 h the reaction mixture is cooled, the solvent is removed in a vacuum, and the residue is mixed with 30 mL 25% ammonia solution and 30 mL ethyl acetate. After separating the organic phase the aqueous mother solution is extracted with 2×30 mL ethyl acetate. The combined organic phases are dried over sodium sulfate, filtered and vacuum concentrated. The product is purified by column chromatography (chloroform:MeOH:ammonia solution=89: 10:1).

Yield: 1.4 g (48.0% of theory) M.p.: 56-63° C. R_{f}: 0.25 (chloroform:MeOH: ammonia solution=89:10:1)

¹H-NMR (DMSO): δ 6.81 (b, 2H), 6.71 (d, 1H), 6.65 (d, 1H), 6.15 (d, 1H), 5.79 (dd, 1H), 4.68 (d, 1H), 4.60 (d, 1H), 4.45 (m, 2H), 4.08 (b, 1H), 3.75 (s, 3H), 3.43 (b, 1H), 2.25 (m, 1H), 2.70 (m, 1H), 2.59 (m, 1H), 2.38 (m, 6H), 2.06 (d, 1H), 1.82 (t, 1H), 1.62 (m, 4H); ¹³C-NMR (DMSO): δ 171.3 (s), 147.9 (s), 144.6 (s) 133.0 (s), 129.7 (s), 129.2 (d), 127.7 (d), 121.5 (d), 112.1 (d), 87.4 (d), 60.6 (d), 56.4 (q), 52.3 (2t), 48.6 (s), 46.5 (t), 44.5 (t), 39.2 (t), 37.2 (t), 32.8 (t), 31.5 (t), 23.9 (2t);

### EXAMPLE 39

### ((4aS,6R,8aS)-4a,5,9,10,11,12-Hexahydro-6-hydroxy-3-methoxy-6H-benzofuro[3a,3,2 -ef][2]benzazepine- I-yl)gamma-oxobutyric acid (la Y₁=OH, Y₂=H, X=H, Z₁=C₄H₅0₃)

2.0 g (−)-norgalanthamine, prepared in accordance with WO-A-017/4820, and 1.53 mL triethylamine and 0.76 g succinic anhydride are stirred in 70 mL tetrahydrofuran at the boiling point. After 1 h the solvent is distilled out, and the residue is mixed with 100 mL 1N HCl and 100 mL ethyl acetate. After separating the organic phase the aqueous phase is extracted with 2×50 mL ethyl acetate. The combined organic phases are dried over sodium sulfate, filtered and vacuum concentrated.

Yield: 1.4 g (51.28% of theory) M.p.: 156-158° C.

R_{f}: 0.7 (ethyl acetate:formic acid=99:1)

¹H-NMR (DMSO): δ 11.95 (b, 1H, OH), 6.81 (b, 1H), 6.71 (dd, 1H), 6.12 (d, 1H), 5.85 (dd, 1H), 4.68 (d, 1H), 4.60 (d, 1H), 4.43 (m, 2H), 4.09 (b, 1H), 3.71 (s, 3H), 2.25 (t, 1H), 2.89 (m, 1H), 2.35 (m, 3H), 2.09 (m, 2H), 1.80 (b, 1H), 1.65 (m, 1H); ¹³C-NMR (DMSO): δ 174.8 (s), 171.2 (s), 147.9 (s), 144.6 (s) 133.0 (s), 129.5 (s), 129.2 (d), 127.7 (d), 121.4 (d), 112.1 (d), 87.3 (d), 60.6 (d), 56.3 (q), 51.9 (t), 48.6 (s), 46.4 (t), 39.7 (t), 31.5 (t), 29.8 (t), 28.5 (t);

### EXAMPLE 40

### 1-((4aS,6R,8aS)-6-Hydroxy-3-methoxy-5,6,9,10-tetrahydro-4aH-[ 1 ]benzofliro[3a,3,2-ef] [2]benzazepine-11(12H)-yl)-2-[2-(2,6-dichloramLino)]phenylethane (Ia Y₁=OH, Y₂=H, X=H, Z₁=CIIHI₃0)

Step 1

6.75 g o-(2,6)-dichloramLino)phenethyl alcohol, prepared in accordance with DE-A-2007700, is added portionwise to a suspension of 7.12 g triphenyl phosphine and 1.36 mL bromine in 100 mL tetrahydofuran at room temperature. After a half hour 100 mL water is added, the mixture is extracted 3 times, each time with 25 mL ethyl acetate, the organic phase is dried over sodium sulfate, filtered and concentrated to 40 mL. After adding 100 mL n-hexane and stirring on an ice bath for one half hour the precipitated triphenyl phosphine oxide is filtered out. The clear filtrate is filtered through a short silica gel column and the solvent is distilled out. There remains 8.2 g of a yellowish oil, which was used directly in the next step without further purification.

Step 2

2.16 g (−)-norgalanthamine HCl, prepared in accordance with WO-A-01/74820, 2.7 g (2,6-dichlorophenyl)-2-(2-bromoethyl)phenylamine and 4.72 g potassium carbonate are stirred in 25 mL acetonitrile at room temperature for 24 h. Then the suspension is poured into 100 mL water and extracted 3 times, each time with 30 mL ethyl acetate. After drying the organic phase over sodium sulfate and subsequent filtration the solvent is distilled out. The resulting 4 g is purified by column chromatography on 200 g silica gel with ethyl acetate, and the product-containing fractions are concentrated by evaporation. Precipitation of the hydrochloride salt of the remaining 800 mg in 15 mL diethyl ether with etheric HCl at 0° C. produces 830 mg, which was recrystallized from 30 mL ethanol.

Yield: 700 mg white crystals (16% of theory) M.p.: 163-165° C. R_{f}: 0.5 (chloroform:MeOH=9:1)

¹H-NMR (CDCl₃): δ 7.42 (b, 1H, NH), 7.35 (d, 2H), 7.13 (dd, 1H), 7.05 (td, 1H), 7.01 (t, 1H), 6.88 (td, 1 H), 6.64 (d, 1H), 6.56 (d, 1H), 6.40 (d, 1H), 6.10 (d, 1H), 6.03 (dd, 1H), 4.63 (m, 1H), 4.22 (d, 1H), 4.16 (m, 1H), 4.03 (d, 1H), 3.78 (s, 3H), 3.48 (td, 1H), 3.29 (dt, 1H), 2.89 (m, 4H), 2.71 (ddd, 1H), 2.42 (b, 1H, OH), 2.13 (td, 1H), 2.01 (ddd, 1H), 1.51 (ddd, 1H); ¹³C-NMR (CDCl₃): δ 145.8 (s), 144.2 (s), 142.8 (s), 137.9 (s), 133.2 (s), 139.5 (s), 130.5 (d), 130.3 (s), 128.8 (d), 128.5 (s), 27.7 (d), 126.8 (d), 126.7 (d), 124.1 (d), 122.3 (d), 120.9 (d), 116.3 (d); 110.9 (d), 88.7 (d), 62.0 (t), 57.3 (t), 55.7 (q), 52.5 (t), 52.0 (t), 48.4 (s), 32.5 (t), 30.7 (t), 29.9 (t);

### EXAMPLE 41

### (4aS,6R,8aS)-4a,5,9,10,11,12-Hexahydro-3-methoxy-11-(4-bromo-benzoyl)-6H -[1]benzofuro[3a,3,2-ef][2]benzazepine-6-ol (Ia Y₁=OH, Y₂=H, X=H, Z₁=CL IH₁30)

2.0 g (−)-norgalanthamine, prepared in accordance with WO-A-01/74820, 4.0 g potassium carbonate and 1.65 g 4-brombenzoic acid chloride are stirred in 70 mL acetonitrile at the boiling point. After 3 h the solvent is distilled out, and the residue is mixed with 100 mL water and 100 mL ethyl acetate. After separating the organic phase the aqueous phase is extracted with 2×50 mL ethyl acetate. The combined organic phases are dried over sodium sulfate, filtered and vacuum concentrated. The product is purified by column chromatography (chloroform:MeOH=95:5)

Yield: 3.3 g (99% of theory) M.p.: 98-112° C. R_{f}: 0.35 (chloroform:MeOH=9:1)

¹H-NMR (CDCl₃): δ 7.51 (d, 2H), 7.13 (d, 2H), 6.65 (d, 1H), 6.24 (d, 1H), 6.08 (td, 1H), 5.98 (d, 1H), 4.90 (b, 1H), 4.68 (b, 1H), 4.42 (s, 1H), 4.18 (d, 1H), 3.85 (s, 3H), 3.45 (m, 1H), 2.73 (dt, 1H), 2.10 (m, 2H), 1.93 (d, 1H), 1.71 (b, 1H); ¹³C-NMR (CDCl₃): δ 170.9 (s), 147.2 (s), 145.0 (s), 135.5 (s), 133.1 (s), 131.8 (2d), 128.9 (2d), 182.5 (d), 128.4 (s), 126.8 (d), 124.3 (s), 121.0 (d), 112.0 (d), 88.8 (d), 62.2 (t), 56.3 (q), 54.9 (t), 48.7 (s), 44.5 (t), 36.6 (t), 30.2 (t);

### EXAMPLE 42

### (4aS,6R,8aS)-11-(4,6-Dichloro-1,3,5,-triazin-2-yl)-3-methoxy-5,6,9,10,11,12-hexahydro-4aH-[1]benzofuro[3a,3,2-ef][2]benzazepine-6-ol (Ia Y₁=OH, Y₂=H, X=H, Z₁=CL IH₁30)

A solution of 1.32 g cyanuric chloride in 32 mL acetone is poured into 70 mL ice water and mixed at 0° C. with

2.0 g (−)-norgalanthamine, prepared in accordance with WO-A-01/74820. The reaction mixture is mixed with 4.0 mL 2N sodium hydroxide solution and stirred at the boiling point. After 40 h the reaction mixture is cooled to room temperature and mixed with 60 mL ethyl acetate. After separating the organic phase the aqueous mother solution is extracted with 2×60 mL ethyl acetate. The combined organic phases are dried over sodium sulfate, filtered and vacuum concentrated. The product is purified by column chromatography (chloroform:MeOH=98:2).

Yield: 1.58 g (51% of theory) M.p.: 245-149° C.

R_{f}: 0.75 (chloroform:MeOH=9:1)

¹H-NMR (DMSO): δ 6.79 (b, 2H), 6.19 (d, 1H), 5.81 (dd, 1H), 5.12 (d, 1H), 4.79 (d, 1H), 4.58 (d, 1H), 4.49 (b, 1H), 4.10 (b, 1H), 3.79 (m, 1H), 3.72 (s, 3H), 2.29 (d, 1H), 2.09 (m, 1H), 1.80 (m, 2H); ¹³C-NMR (DMSO): δ 170.0 (s), 169.9 (s), 164.3 (s), 147.9 (s), 144.7 (s) 132.9 (s), 129.4 (d), 127.6 (d), 127.5 (s), 121.8 (d), 112.2 (d), 87.0 (d), 60.4 (d), 56.4 (q), 52.1 (t), 48.5 (s), 46.6 (t), 40.0 (t), 31.4 (t);

### EXAMPLE 43

### (4aS,6R,8aS)-11-(4-Brombenzyl)-4a,5,9,10,11,12-hexahydro-3-methoxy-6H -[1]benzofuro[3a,3,2-ef][2]benzazepine-6-ol (Ia Y₁=OH, Y₂=H, X=H, Z₁=C₇H₆Br)

3.0 g (−)-norgalanthamine, prepared in accordance with WO-A-01/74820, 3.0 g potassium carbonate and 2.5 g 4-bromobenzyl bromide are stirred in 70 mL acetonitrile at room temperature. After 24 h the solvent is removed under a vacuum and the residue is mixed with 100 mL water and 40 mL ethyl acetate. After separating the organic phase the aqueous mother solution is extracted with 2×40 mL ethyl acetate. The combined organic phases are dried over sodium sulfate, filtered and vacuum concentrated. The product is purified by column chromatography (chloroform:MeOH=99:1).

Yield: 3.0 g (70.2% of theory) M.p.: 148-149° C. R_{f}: 0.8 (chloroform:MeOH=9:1)

¹H-NMR (CDCl₃): δ 7.53 (d, 2H), 7.18 (d, 2H), 6.65 (d, 1H), 6.40 (d, 1H), 6.15 (d, 1H), 6.03 (d, 1H), 4.68 (b, 1H), 4.12 (m, 1H), 3.85 (s, 3H), 3.66 (d, 1H), 3.61 (s, 2H), 3.41 (t, 1H), 3.18 (d, 1H), 2.71 (dd, 1H), 2.44 (d, 1H), 2.15 (dd, 1H), 2.03 (dd, 1H), 1.65 (d, 1H); ¹³C-NMR (CDCI₃): δ 146.3 (s), 144.5 (s), 138.4 (s), 133.7 (s), 131.8 (2d), 131.0 (2d), 129.8 (s), 128.1 (d), 127.2 (d), 122.5 (d), 121.2 (s), 111.6 (d), 89.2 (d), 62.5 (t), 57.7(t), 56.3 (q), 56.0 (t), 52.2 (t), 48.9 (s), 33.9 (t), 30.4 (t);

### EXAMPLE 44

### Ethyl-2-((4aS,6R,8aS)-6-hydroxy-3-methoxy-5,6,9,10-tetrahydro-4aH-[1 ]benzofuro [3a,3,2-ef][2]benzazepine-11(12H)-yl) acetate (Ia Y,=OH, Y₂=H, X=H, Z₁=C₄H₇0₂)

2.0 g (−)-norgalanthamine, prepared in accordance with WO-A-01/74820, 4.0 g potassium carbonate and 1.0 m]L bromoacetic acid ethyl ester are stirred in 50 mL tetrahydrofuran at room temperature. After 16 h the precipitate is filtered out, and the filtrate is mixed with 100 mL water and 50 mL ethyl acetate. After separating the organic phase the aqueous mother solution is extracted with 2×50 mL ethyl acetate. The combined organic phases are dried over sodium sulfate, filtered and vacuum concentrated. The product is purified by column chromatography (pure ethyl acetate).

Yield: 1.46 g (55.5% of theory) M.p.: 75-78° C. R_{f}: 0.8 (ethyl acetate)

¹H-NMR (DMSO): δ 6.69 (d, 1H), 6.49 (d, 1H), 6.08 (d, 1H), 5.80 (dd, 1H), 4.49 (b, 1H), 4.21 (m, 2H), 4.08 (m, 2H), 3.75 (s, 3H), 3.68 (m, 1H), 3.32 (m, 2H), 2.23 (d, 1H), 3.00 (d, 1H), 2.28 (d, 1H), 2.07 (td, 1H), 1.93 (t, 1H), 1.58 (d, 1H), 1.18 (t, 3H); ¹³C-NMR (DMSO): δ 171.4 (s), 147.1 (s), 144.2 (s), 133.6 (s), 130.1 (s), 129.2 (d), 127.8 (d), 121.7 (d), 112.2 (d), 87.7 (d), 60.7 (d), 60.6 (t), 58.0 (t), 56.3 (q), 54.6 (t), 52.5 (t), 48.6 (s), 35.1 (t), 31.9 (t), 15.0 (q);

### EXAMPLE 45

### 2-((4aS,6R,8aS)-6-Hydroxy-3-methoxy-5,6,9,10-tetrahydro-4aH-[ 1 ]benzofuro[3a,3,2-ef][2]benzazepine-11(12H)-yl)acetic acid (Ia Y₁=OH, Y₂=H, X=H, Z₁=C₂H₃0₂)

Step 1

2.0 g (−)-norgalanthamine, prepared in accordance with WO-A-01/74820, 4.0 g potassium carbonate and 1.0 mL bromoacetic acid ethyl ester are stirred in 50 mL tetrahydrofuran at room temperature. After 16 h the precipitate is separated, the solvent is removed under a vacuum and the residue is mixed with 100 mL water and 50 mL ethyl acetate. After separating the organic phase the aqueous mother solution is extracted with 2×50 mL ethyl acetate. The combined organic phases are dried over sodium sulfate, filtered and vacuum concentrated. One obtains 2.8 g foam-like material.

Step 2

1.6 g of the product obtained in Step 1 is dissolved in 24 mL ethanol, mixed with 3.2 mL 2N sodium hydroxide solution and stirred at room temperature. After 30 min the clear solution is mixed with 4.8 g IRA-120 ion exchanger and stirred for another 10 min. The ion exchanger is filtered out, the filtrate is vacuum concentrated and the residue is recrystallized from a mixture of methanol and tert-butyl methyl ether (MTBE).

Yield: 0.8 g white powder M.p.: 144-161° C. R_{f}: 0.2 (chloroform:MeOH=6:4)

¹H-NMR (DMSO): δ 6.69 (d, 1H), 6.49 (d, 1H), 6.08 (d, 1H), 5.80 (dd, 1H), 4.49 (b, 1H), 4.21 (m, 2H), 4.08 (m, 2H), 3.75 (s, 3H), 3.68 (mn, 1H), 3.32 (mn, 2H), 2.23 (d, 1H), 3.00 (d, 1H), 2.28 (d, 1H), 2.07 (td, 1H), 1.93 (t, 1H), 1.58 (d, 1H), 1.18 (t, 3H); ¹³C-NMR (DMSO): δ 171.4 (s), 147.1 (s), 144.2 (s), 133.6 (s), 130.1 (s), 129.2 (d), 127.8 (d), 121.7 (d), 112.2 (d), 87.7 (d), 60.7 (d), 60.6 (t), 58.0 (t), 56.3 (q), 54.6 (t), 52.5 (t), 48.6 (s), 35.1 (t), 31.9 (t), 15.0 (q);

### EXAMPLE 46

### (4aS,6R,8aS)-4a,5,9,10,11,12-Hexahydro-3-methoxy-11-(2-methyl-prop-2-enyl)-6H -[1]benzofuro[3a,3,2-ef][2]benzazepine-6-ol, hydrochloride (Ia Y₁=OH, Y₂=H, X=H, Z₁=C₂H₃0₂)

2.0 g (−)-norgalanthamine, prepared in accordance with WO-A-01/74820, 2.02 g potassium carbonate, 1.27 g potassium iodide and 0.85 mL 3-chloro-2-methyl-1-propene are stirred in 80 mL acetone at reflux temperature. After 4 h the reaction mixture is cooled to room temperature, the solvent is vacuum distilled out, and the residue is mixed with 200 mL 2N HCl and 50 mL ethyl acetate. After separation the organic phase is discarded. The aqueous phase is made basic with 30% sodium hydroxide solution and extracted with 3×100 mL ethyl acetate. The combined organic phases are dried over sodium sulfate, filtered, and vacuum concentrated. The product is purified by column chromatography (chloroform:MeOH=95:5). The resulting oil is dissolved in ethanol and the hydrochloride salt is precipitated with etheric HCl.

Yield: 2.38 g (99% of theory) M.p.: 233-234° C. R_{f}: 0.8 (chloroform:MeOH:ammonia solution=89:10:1)

¹H-NMR (DMSO): δ 6.88 (d, 1H), 6.70 (d, 1H), 6.17 (d, 1H), 5.93 (d, 1H), 5.39 (d, 1H), 5.20 (d, 1H), 4.62 (m, 2H), 4.28 (m, 1H), 4.12 (b, 1H), 3.95 (b, 1H), 3.81 (s, 3H), 3.62 (m, 3H), 2.29 (d, 1H), 2.10 (d, 2H), 1.93 (d, 3H), 1.58 (d, 1H); ¹³C-NMR (DMSO): δ 147.4 (s), 145.8 (s), 136.5 (s), 134.1 (s), 130.9 (d), 126.3 (d), 123.5 (d), 122.6 (s), 112.8 (d), 87.4 (d), 67.9 (t), 64.2 (t), 57.7 (d), 56.6 (t), 56.4 (q), 50.5 (t), 47.6 (s), 31.9 (t), 26.0 (t), 22.2 (q);

### EXAMPLE 47

### Ethyl-3-((4aS,6R,8aS)-6-hydroxy-3-methoxy-5,6,9,10-tetrahydro-4aH-[ 1 ]benzofuro [3a,3,2-ef][2]benzazepine-11(12H)-yl)propanoate, hydrochloride (Ia Y₁=OH, Y₂=H, X=H, Z₁=C₅H₉0₂)

0.55 g (−)-norgalanthamine, prepared in accordance with WO-A-01/74820, is stirred with 0.3 mL ethyl acrylate in 20 mL abs. ethanol at reflux temperature. After 72 h the solvent is distilled out and the product is purified by column chromatography (chloroform:MeOH=95:5). The resulting oil is dissolved in chloroform and the hydrochloride salt is precipitated with etheric HCl.

Yield: 0.5 g (60.6% of theory) R_{f}: 0.6 (chloroform:MeOH=95:5)

¹H-NMR (DMSO): δ 6.88 (d, 1H), 6.70 (d, 1H), 6.17 (d, 1H), 5.93 (d, 1H), 5.39 (d, 1H), 5.20 (d, 1H), 4.62 (m, 2H), 4.28 (m, 1H), 4.12 (b, 1H), 3.95 (b, 1H), 3.81 (s, 3H), 3.62 (m, 3H), 2.29 (d, 1H), 2.10 (d, 2H), 1.93 (d, 3H), 1.58 (d, 1H); ¹³C-NMR (DMSO): δ 147.4 (s), 145.8 (s), 136.5 (s), 134.1 (s), 130.9 (d), 126.3 (d), 123.5 (d), 122.6 (s), 112.8 (d), 87.4 (d), 67.9 (t), 64.2 (t), 57.7 (d), 56.6 (t), 56.4 (q), 50.5 (t), 47.6 (s), 31.9 (t), 26.0 (t), 22.2 (q);

### EXAMPLE 48

### 1-[(4aS,6R,8aS)-6-Hydroxy-3-methoxy-5,6,9,10-tetrahydro-4aH-[1]benzofuro[3a,3,2-ef] [2]benzazepine-11(12H)-yl]-2-(4-morpholinyl)ethan-1-one (Ia Y₁=OH, Y₂=H, X=H, Z₁=C₈H₁₄N₃0)

3.0 g (−)-norgalanthamine, prepared in accordance with WO-A-01/74820, 1.86 mL triethylamine and 0.9 mL chroroacetyl chloride are stirred in 150 mL tetrahydrofuran at 0° C. After 10 min the reaction mixture is mixed with 3.0 g potassium carbonate and 1.2 mL morpholine and stirring is continued at 90° C. After 60 h the solvent is distilled out, and the residue is mixed with 50 mL water and 50 mL chloroform. After separating the organic phase the aqueous phase is extracted with 2×30 mL chloroform. The combined organic phases are dried over sodium sulfate, filtered and vacuum concentrated. The product is purified by column chromatography (chloroform:MeOH=98:2).

Yield: 2.5 g (56.8% of theory) M.p.: 92-101° C. R_{f}: 0.45 (chloroform:MeOH=9:1)

¹H-NMR (CDCI₃): δ 6.67 (m, 2H), 6.01 (m, 2H), 4.99 (d, 1H), 4.63 (d, 1H), 4.55 (b, 1H), 4.37 (d, 1H), 4.12 (b, 1H), 3.82 (s, 3H), 3.67 (m, 4H), 3.25 (d, 1H), 3.14 (m, 2H), 3.00 (d, 1H), 2.48 (m, 4H), 2.01 (dd, 1H), 1.88 (t, 1H), 1.75 (d, 1H); ¹³C-NMR (CDCl₃): δ 169.2 (s), 147.3 (s), 145.0 (s), 132.9 (s), 128.9 (s), 128.8 (d), 126.8 (d), 122.4 (d), 111.7 (d), 88.7 (d), 67.1 (2t), 62.1 (d), 56.3 (q), 54.1 (2t), 52.7 (t), 48.7 (s), 45.5 (t), 38.9 (t), 36.1 (t), 30.3 (t);

### EXAMPLE 49

### 1-[(4aS,6R,8aS)-6-Hydroxy-3-methoxy-5,6,9,10-tetrahydro-4aH-[ 1 ]benzofuro[3a,3,2-ef] [2]benzazepine-11(12H)-yl]-2-(diethylamino)ethan-1-one (Ia Y₁=OH, Y₂=H, X=H, Z₁=C₆H₁₂NO) 3.0 g (−)-norgalanthamine, prepared in accordance with WO-A-01/74820, 1.86 mL triethylamine and 0.9 mL chroroacetyl chloride are stirred in 150 mL tetrahydrofuran at 0° C. After 10 min the reaction mixture is mixed with 3.0 g potassium carbonate and 0.75 mL diethylamine and stirring is continued at 90° C. After 24 h the solvent is distilled out, and the residue is mixed with 50 mL water and 50 mL chloroform. After separating the organic phase the aqueous phase is extracted with 2×30 mL chloroform. The combined organic phases are dried over sodium sulfate, filtered, and vacuum concentrated. The product is purified by column chromatography (chloroform:MeOH=95:5).

Yield: 2.0 g (48.5% of theory) M.p.: 114-126° C. R_{f}: 0.45 (chloroform:MeOH=9:1)

¹H-NMR (CDCl₃): δ 6.68 (mn, 2H), 6.01 (m, 2H), 5.19 (d, 1H), 4.60 (m, 1H), 4.33 (d, 1H), 4.09 (b, 1H), 3.78 (s, 3H), 3.37 (d, 1H), 3.21 (m, 1H), 2.95 (d, 1H), 2.64 (m, 3H), 2.49 (m, 3H), 2.03 (dd, 1H), 1.92 (t, 1H), 1.75 (d, 1H), 1.01 (m, 6H); ¹³C-NMR (CDCl₃): δ 170.8 (s), 147.2 (s), 145.0 (s), 132.9 (s), 129.1 (s), 128.6 (d), 126.9 (d), 121.0 (d), 111.7 (d), 88.7 (d), 62.3 (d), 56.3 (t), 56.2 (q), 52.4 (t), 48.7 (2t), 47.8 (s), 38.9 (t), 36.1 (t), 30.3 (t), 12.0 (2q);

### EXAMPLE 50

(4aS,6R,8aS)-4a,5,9,10,11,12-Hexahydro-3-methoxy-11-[3-(1-piperidinyl)butyl] -6H-benzofuro[3a,3,2-ef][2]benzazepine-6-ol, (+) Di-O-p-toluoyl tartrate, (Ia Y₁=OH, Y₂=H, X=H, Z₁=C₉H₁₉N)

Step 1:

4.1 g 4-brombutanoic acid piperidine amide (17.5 mmol) is dissolved in 100 mL acetonitrile. 3.8 g (−)norgalanthamine hydrochloride, prepared in accordance with WO-A-01/74820, and 9.7 g potassium carbonate are added to the solution and the solution is stirred for 30 h at 80° C. After filtering out the potassium carbonate the solvent is distilled out and the residue is taken up in 100 mL toluene and 100 mL 1N HCl. The aqueous phase is made basic with 30% sodium hydroxide solution and extracted by shaking 3 times, each time with 40 mL ethyl acetate. The combined organic phase is dried over sodium sulfate, filtered and concentrated. The resulting 4.4 g of brown oil is purified by column chromatography on 200 g silica gel with the eluent chloroform:methanol=98:2.

Yield: 1.6 g (30% of theory)

Step 2:

3.6 g Step 1 is dissolved in 50 mL tetrahydrofuran, cooled to 0° C. and 705 mg lithium aluminium hydride is added in portions over 20 min. After this has been completed, the mixture is heated to room temperature and stirred for 1.5 h. The reaction is quenched dropwise with water, the resulting precipitate is filtered out and washed with 10 mL tetrahydrofuran. After drying with sodium sulfate the solution is filtered and the solvent is removed. The resulting 3.4 g are chromatographed on 200 g silica gel with the eluent chloroform:methanol:ammonia solution=90:9:1. The resulting 2.4 g is dissolved in 80 mL ethyl acetate and precipitated with 2.5 g (+)-di-p-toluyl-D-tartaric acid in 30 mL ethyl acetate, filtered and washed with 10 mL ethyl acetate.

Yield: 4.4 g colorless crystals (78.7% of theory)

¹H-NMR (CDCl₃): δ 6.67 (d, 1H), 6.61 (d, 1H), 6.12 (d, 1H), 6.01 (dd, 1H), 4.63 (m, 1H), 4.15 (m, 2H), 3.88 (s, 3H), 3.82 (d, 1H), 3.35 (d, 1H), 3.18 (d, 1H), 2.70 (dd, 1H), 2.49 (m, 4H), 2.31 (m, 4H), 2.02 (m, 4H), 1.71 (m, 4H), 1.49 (m, 5H); ¹³C-NMR (CDCl₃): δ 146.1 (s), 144.4 (s), 133.5 (s), 129.9 (s), 127.9 (d), 127.4 (d), 122.4 (d), 111.5 (d), 89.1 (d), 66.1 (t), 62.5 (d), 59.7 (t), 58.3 (t), 56.2 (q), 54.9 (2t), 51.9 (t), 48.8 (s), 33.4 (t), 30.3 (t), 26.3 (2t), 26.0 (t), 25.1 (t), 24.9 (t);

### EXAMPLE 51

### 3-((4aS,6R,8aS)-1-Bromo-6-hydroxy-3-methoxy-5,6,9,10-tetrahydro-4aH-[1 ]benzofuro [3a,3,2-ef][2]benzazepine-11(12H)-yl)propanenitrile (Ia Y₁=OH, Y₂=H, X=H, Z₁=C₃H₄N)

2.0 g (−)-norgalanthamine, prepared in accordance with WO-A-01/74820, 2.0 g CaCl₂ and 0.5 mL acrylonitrile are stirred in 200 mL ethanol at the boiling point. After 8 h the solvent is distilled out, the residue is taken up in 500 mL 2N HC1 and extracted with 3×200 mL ethyl acetate. The aqueous mother solution is made basic with 25% ammonia solution and extracted with 3×200 mL methylene chloride. The combined organic phases are dried over sodium sulfate, filtered, and vacuum concentrated. The product is purified by column chromatography (chloroform:MeOH=9:1).

Yield: 1.7 g (62% of theory) M.p.: 69-72° C. R_{f}: 0.45 (chloroform:MeOH:ammonia solution=90:9:1)

¹H-NMR (CDCl₃): δ 6.67 (d, 1H), 6.60 (d, 1H), 6.06 (d, 1H), 5.98 (dd, 1H), 4.69 (b, 1H), 4.21 (d, 1H), 4.10 (m, 1H), 3.82 (s, 3H), 3.79 (d, 1H), 3.45 (t, 1H), 3.27 (d, 1H), 2.80 (m, 2H), 2.67 (dd, 1H), 2.43 (m, 2H), 1.99 (m, 2H), 1.59 (d, 1H); ¹³C-NMR (CDCl₃): δ 146.5 (s), 144.8 (s), 133.4 (s), 129.0 (s), 128.3 (d), 126.9 (d), 122.4 (d), 119.3 (s), 111.7 (d), 89.0 (d), 62.3 (d), 57.4 (t), 56.3 (q), 52.1 (t), 48.9 (s), 47.0 (t), 33.5 (t), 30.4 (t), 7.2 (t);

### EXAMPLE 52

### (4aS,6R,8aS)-11-((3-Dimethylamino)propyl)-3-methoxy-5,6,9,10,11,12-hexahydro-4aH -[1]benzofuro[3a,3,2-ef][2]benzazepine-6-ol (Ia Y₁=OH, Y₂=H, X=H, Z₁=C₅NH₂N)

3.0 g (−)-norgalanthamine HCl, prepared in accordance with WO-A-01/74820, 5.0 g potassium carbonate and 2.1 g 3-dimethylaminopropyl chloride HCl are stirred in 70 mL acetonitrile at the boiling point. After 28 h the precipitate is filtered out and the solvent is removed in a vacuum. The product is purified by column chromatography (chloroform:MeOH=9:1).

Yield: 2.35 g brown oil (59.8% of theory) R_{f}: 0.35 (chloroform:MeOH=9:1)

¹H-NMR (DMSO): δ 6.67 (d, 1H), 6.60 (d, 1H), 6.11 (d, 1H), 5.94 (dd, 1H), 4.59 (b, 1H), 4.13 (m, 2H), 3.82 (s, 3H), 3.78 (d, 1H), 3.21 (m, 11H), 2.52 (m, 2H), 2.29 (m, 1H), 2.05 (d, 1H), 2.65 (m, 2H), 1.51 (d, 1H); ¹³C-NMR (DMSO): δ 146.3 (s), 144.2 (s), 133.5 (s), 129.8 (s), 128.0 (d), 127.5 (d), 122.2 (d), 111.6 (d), 88.7 (d), 61.9(d), 58.0(t), 57.8 (t), 56.2 (q), 52.0(t), 50.0 (t), 48.7 (s), 45.7 (2q), 33.4 (t), 30.7 (t), 25.8 (t);

### EXAMPLE 53

### (4aS,6R,8aS)-N11-Cyclohexyl-6-hydroxy-3-methoxy-5,6,9,10-tetrahydro-4aH -[ I ]benzofuro[3a,3,2-ef][2]benzazepine-11 (1 2H)-carbonic acid isopropylamide (Ia Y₁=OH, Y₂=H, X=H, Z₁=C₇H₁₂NO)

2.0 g (−)-norgalanthamine, prepared in accordance with WO-A-01/74820, and 0.92 g cyclohexyl isocyanate are dissolved in 100 mL toluene and stirred at the boiling point. After 5 h the solvent is distilled out in a vacuum, and the residue is mixed with 200 mL 2 N HCl and 100 mL diethyl ether. After separating the organic phase the aqueous phase is made basic with 25% ammonia solution and extracted with 3×100 mL ethyl acetate. The combined organic phases are dried over sodium sulfate, filtered and the solvent is distilled out under a vacuum. The residue is crystallized from ethanol.

Yield: 1.97 g (67.5% of theory) M.p.: 168-170° C. R_{f}: 0.6 (chloroform:MeOH=9:1)

¹H-NMR (CDCl₃): δ 6.69 (d, 1H), 6.62 (d, 1H), 5.95 (m, 2H), 4.57 (b, 1H), 4.46 (d, 1H), 4.31 (d, 2H), 4.12 (b, 1H), 3.80 (s, 3H), 3.41 (m, 1H), 3.32 (t, 1H), 2.63 (d, 1H), 2.03 (dd, 1H), 1.91 (d, 2H), 1.70 (d, 2H), 1.55 (m, 2H), 1.25 (m, 2H), 1.09 (m, 2H), 0.95 (m, 2H); ¹³C-NMR (CDCl₃): δ 156.9 (s), 147.3 (s), 145.0 (s), 132.9 (s), 129.6 (s), 128.4 (d), 126.9 (d), 120.7 (d), 111.5 (d), 88.8 (d), 62.2 (d), 56.3 (q), 52.0 (t), 49.6 (d), 48.9 (t), 46.0 (s), 36.9 (t), 34.2 (t), 33.9 (t), 30.2 (t), 6.0 (t), 25.2 (t), 25.1 (t);

### EXAMPLE 54

### 1-[(4aS,6R,8aS)-6-Hydroxy-3-methoxy-5,6,9,10-tetrahydro-4aH-[ 1 ]benzofuro[3a,3,2-ef] [2]benzazepine-11(1 2H)-yl] -2-chloroethan-1-one (Ia Y=OH, Y₂=H, X=H, Z I=C₂H₂C₁₀)

A solution of 3.0 g (−)-norgalanthamine, prepared in accordance with WO-A-01/74820, and 1.9 mL triethylamine in 150 mL tetrahydrofuran are mixed with 0.93 mL chloroacetyl chroride at 0° C. After 10 min the solvent is removed in a vacuum, the residue is mixed with 100 mL water and 10 mL 2 N hydrochloric acid and extracted with 3×30 mL diethyl ether. The combined organic phases are dried over sodium sulfate, filtered and the solvent is vacuum distilled out.

Yield: 1.42 g (37.1% of theory) M.p.: 88-90° C. R_{f}: 0.8 (chloroform:MeOH=9:1)

¹H-NMR (CDCl₃): δ 6.73 (m, 2H), 6.02 (m, 2H), 4.69 (d, 1H), 4.65 (d, 1H), 4.52 (d, 1H), 4.19 (d, 1H), 4.09 (m, 2H), 3.95 (d, 1H), 3.85 (s, 3H), 3.30 (t, 1H), 2.73 (d, 1H), 2.09 (dd, 1H), 1.91 (d, 1H), 1.71 (d, 1H); ¹³C-NMR (CDCl₃): δ 166.5 (s), 147.5 (s), 145.5 (s), 132.8 (s), 129.0 (s), 128.2 (d), 126.5 (d), 122.6 (d), 111.7 (d), 88.8 (d), 62.2 (d), 56.4 (q), 53.4 (t), 48.7 (d), 46.0 (s), 41.9 (t), 35.9 (t), 30.2 (t);

### EXAMPLE 55

### (4aR,6S,8aR)-6-Hydroxy-3-methoxy-11-methyl-4a,5,9,1 0-tetrahydro-6H-benzofuro [3a,3,2-ef] [2]benzazepinium bromide

A solution of 2.0 g (+)-galanthamine, prepared in accordance with Kametani, Heterocycles 4, 1111, 1976, in 20 mL chloroform is vigorously stirred and mixed with a solution of 1.26 g N-bromosuccinimide in 20 mL chloroform that is added by drops at room temperature. After 1 h the precipitate that forms is separated, washed with chloroform and dried in a vacuum dryer at 50° C. The product is recrystallized from ethanol.

Yield: 2.28 g (90.2% of theory) M.p.: 223-229° C. R_{f}: 0.2 (chloroform:MeOH:ammonia solution=90:9:1)

¹H-NMR (DMSO): δ 9.15 (s, 1H), 7.57 (d, 1H), 7.20 (d, 1H), 5.89 (dd, 1H), 5.72 (d, 1H), 4.67 (b, 1H), 4.61 (d, 1H), 4.13 (m, 3H), 3.95 (s, 3H), 3.80 (s, 3H), 2.35 (d, 1H), 2.15 (m, 2H); ¹³C-NMR (DMSO): δ 167.3 (d), 151.3 (s), 146.2 (s), 136.9 (s), 133.0 (d), 129.8 (d), 126.4 (d), 115.0 (s), 112.9 (d), 86.9 (d), 58.9 (d), 56.4 (q), 54.0 (t), 51.5 (t), 45.9 (q), 40.7 (t), 31.1 (t), 29.7 (t);

### EXAMPLE 56

### (4aR, 6R, 8aR)-6-Hydroxy-3-methoxy-11-methyl-4a,5,9,10-tetrahydro-6H-benzofuro [3a, 3, 2-ef][2]benzazepinium bromide

1.35 g N-bromosuccinimide is dispensed at room temperature into a vigorously stirred solution of 2.0 g (+)-epigalanthamine, prepared in accordance with J. Chem. Soc. 806, 1962, in 80 mL chloroform. After 1 h the precipitate that has formed is separated, washed with chloroform and dried in a vacuum dryer at 50° C. The product is recrystallized from ethanol.

Yield: 2.09 g (82.7% of theory) M.p.: 236-244° C. R_{f}: 0.2 (chloroform:MeOH: ammonia solution=90:9:1)

¹H-NMR (DMSO): δ 9.15 (s, 1H), 7.58 (d, 1H), 7.20 (d, 1H), 5.85 (dd, 1H), 5.74 (d, 1H), 5.15 (d, 1H), 4.79 (b, 1H), 4.30 (m, 1H), 4.13 (m, 2H), 3.93 (s, 3H), 3.80 (s, 3H), 2.55 (d, 1H), 2.20 (m, 1H), 1.73 (dt, 1H); ¹³C-NMR (DMSO): δ 168.1 (d), 152.1 (s), 147.3 (s), 138.1 (s), 135.4 (d), 133.9 (d), 126.9 (d), 116.0 (s), 113.9 (d), 88.9 (d), 61.7 (d), 57.3 (q), 55.1 (t), 52.3 (t), 47.1 (q), 41.0 (t), 32.3 (t), 31.7 (t);

### EXAMPLE 57

### 4aS,6R,8aS)-1-Bromo-4a,5,9,10,11,12-hexahydro-11-(2-(morpholin-4-yl)-ethyl) -3-methoxy-6H-[1]benzofuro[3a,3,2-ef][2]benzazepine-6-ol (Ia Y₁=OH, Y₂=H, X=Br, Z₁=C₆H₁₂NO)

2.0 g (−)-bromonorgalanthamine (Ia Y₁=OH, Y₂=H, X=Br, Z₁=H), 2.35 g potassium carbonate and 1.11 g N-(2-chloroethyl)morpholine hydrochloride are stirred in 30 mL acetonitrile at reflux temperature. After 48 h the reaction mixture is cooled to room temperature, the solvent is vacuum distilled out, and the residue is mixed with 200 mL 2N HCl and 40 mL ethyl acetate. After separation the organic phase is discarded. The aqueous phase is made basic with ammonia solution and extracted with 3×40 mL ethyl acetate. The combined organic phases are dried over sodium sulfate, filtered, and vacuum concentrated. The product is purified by column chromatography (chloroform:MeOH=95:5).

Yield: 1.39 g white foam (53% of theory) R_{f}: 0.2 (chloroform:MeOH:ammonia solution=90:9:1)

¹H-NMR (DMSO): δ 6.91 (s, 1H), 6.15 (d, 1H), 6.03 (dd, 11H), 4.51 (b, 1H), 4.41 (d, 1H), 4.16 (b, 1H), 4.05 (d, 1H), 3.85 (s, 3H), 3.71 (t, 4H), 3.39 (t, 1H), 3.15 (d, 1H), 2.69 (m, 3H), 2.51 (m, 5H), 2.03 (m, 3H), 1.55 (d, 1H); ¹³C-NMR (CDCl₃): δ 145.9 (s), 144.7 (s), 134.6 (s), 128.5 (d), 128.4 (s), 127.4 (d), 116.3 (d), 114.9(s), 89.2 (d), 67.3 (2t), 62.3 (d), 57.3 (2t), 56.6(q), 56.6(t), 54.6 (2t), 52.6 (t), 49.4 (t), 33.6 (t), 30.2 (t);

### EXAMPLE 58

(4aR,6R,8aRS)-1-Bromo-4a,5,9,10,11,12-hexahydro-11-(2-(morpholin-4-yl)-ethyl) -3-methoxy-6H-[1]benzofuro[3a,3,2-ef][2]benzazepine-6-ol (Ib Y₁=OH, Y₂=H, X=Br,

### Z₁=C₆H₁₂NO)

3.0 g (+)-bromonorgalanthamine (Ib Y₁=OH, Y₂=H, X=Br, Z₁=H), 4.8 g potassium carbonate and 1.47 g N-(2-chloroethyl)morpholine hydrochloride are stirred in 30 mL acetonitrile at reflux temperature. After 22 h the reaction mixture is cooled to room temperature, the solvent is vacuum distilled out, and the residue is mixed with 100 mL water and 40 mL ethyl acetate. After separating the aqueous phase is extracted with 3×40 mL ethyl acetate. The combined organic phases are dried over sodium sulfate, filtered and vacuum concentrated. The product is purified by column chromatography (eluent 96% ethanol).

Yield: 1.9 g (48% of theory) M.p.: 58-64° C. R_{f}: 0.2 (96% ethanol)

¹H-NMR (DMSO): δ 6.99 (s, 1H), 6.12 (d, 1H), 5.82 (dd, 1H), 4.55 (b, 1H), 4.37 (b, 1H), 4.20 (d, 1H), 4.05 (m, 2H), 3.79 (s, 3H), 3.51 (m, 4H), 3.32 (d, 1H), 3.28 (d, 1H), 2.99 (d, 1H), 2.51 (m, 2H), 2.35 (m, 4H), 2.25 (d, 1H), 2.00 (m, 2H), 1.49 (d, 1H); ¹³C-NMR (CDCI₃): δ 146.9 (s), 144.6 (s), 134.9 (s), 129.6 (d), 128.7 (s), 127.4 (d), 116.3 (d), 113.3 (s), 87.8 (d), 67.1 (2t), 60.5 (d), 57.1 (2t), 56.7 (q), 55.9 (t), 54.5 (2t), 52.2 (t), 49.4 (t), 34.0 (t), 31.7 (t);

### EXAMPLE 59

### (4aS,8aS)-A^{5 6}-4a,5,9,10,11,12-Hexahydro-11-methyl-3-methoxy-6-phenyl-6H -[1]benzofuro[3a,3,2-ef][2]benzazepine (Ic Y₃=Phenyl, X=H, Z₂=CH₃)

3.35 mL bromobenzene is added by drops to a mixture of 1.16 g magnesium in 15 mL tetrahydrofuran. The resulting reaction mixture is vigorously stirred for 1 h, mixed with a solution of 3.0 g (−)-narwedine, prepared in accordance with EP-A-0787115, in 50 mL tetrahydrofuran with continued stirring. After 2 h 60 mL water and 40 mL 2N HCl are added by drops to the reaction mixture and the resulting suspension is stirred at 60° C. After 50 min the reaction mixture is cooled to room temperature, the pH is adjusted to 9 with an ammonia solution and the mixture is extracted with 3×100 mL ethyl acetate. The combined organic phases are dried over sodium sulfate, filtered and vacuum concentrated. The product is purified by column chromatography (chloroform:MeOH=99:1).

Yield: 1.8 g colorless foam (49% of theory) R_{f}: 0.35 (chloroform:MeOH=99:1)

¹H-NMR (CDCl₃): δ 7.50 (d, 2H), 7.39 (m, 3H), 6.65 (b, 2H), 6.42 (d, 1H), 6.33 (m, 2H), 5.00 (d, 1H), 4.28 (d, 1H), 3.89 (s, 3H), 3.75 (d, 1H), 3.51 (t, 1H), 3.09 (d, 1H), 2.41 (s, 3H), 2.13 (dt, 1H), 1.79 (dd, 1H); ¹³C-NMR (CDCl₃): δ 148.2 (s), 144.3 (s), 139.8 (s), 139.5 (s), 131.6 (s), 131.2 (d), 130.0 (s), 129.0 (2d), 128.6 (d), 126.6 (2d), 123.2 (d), 122.2 (d), 116.1 (d), 110.7 (d), 86.5 (d), 60.8 (t), 56.2 (q), 54.5 (t), 48.7 (t), 42.2 (q), 35.4 (t);

### EXAMPLE 60

### (4aS,8aS)-A⁵,⁶-4a,5,9,10,11,12-hexahydro-6, 11-dimethyl-3-methoxy-6H -[1]benzofuro[3a,3,2-ef][2]benzazepine (Ic Y₃=CH₃, X=H, Z₂=CH₃)

A solution of 2.04 g (−)-narwedine, prepared in accordance with EP-A-0787115, in 60 mL tetrahydrofuran is mixed dropwise with 10.0 mL methyl magnesium bromide in diethyl ester and stirred at room temperature. After 45 min 60 mL water and 20 mL 2N HCl are added by drops to the reaction mixture and the resulting suspension is stirred at 60° C. After 50 min the reaction mixture is cooled to room temperature, the pH is adjusted to 9 with an ammonia solution and the mixture is extracted with 3×100 mL ethyl acetate. The combined organic phases are dried over sodium sulfate, filtered and vacuum concentrated. The product is purified by column chromatography (chloroform:MeOH=98:2).

Yield: 2.18 g colorless foam (72% of theory) R_{f}: 0.5 (chloroform:MeOH=99:1)

¹H-NMR (CDCl₃): δ 6.57 (m, 2H), 6.01 (d, 1H), 5.87 (d, 1H), 5.69 (m, 1H), 4.80 (d, 1H), 4.15 (d, 1H), 3.80 (s, 3H), 3.68 (d, 1H), 3.34 (t, 1H), 3.01 (d, 1H), 2.41 (s, 3H), 2.09 (dt, 1H), 1.91 (s, 3H), 1.71 (dd, 1H); ¹³C-NMR (CDCl₃): δ 148.2 (s), 144.2 (s), 136.6 (s), 131.8 (s), 130.2 (s), 130.0 (d), 125.2 (d), 121.9 (d), 115.3 (d), 110.5 (d), 88.9 (d), 60.8 (t), 56.2 (q), 54.6 (t), 48.5 (t), 42.4 (q), 35.4 (t), 22.2 (q);

### EXAMPLE 61

### (4aS,8aS)-A⁵,⁶-4a,5,9, 10,11,1 2-Hexahydro-6-(isopropyl)-11-methyl-3-methoxy-6H -[1]benzofuro[3a,3,2-ef][2]benzazepine (Ic, Y₃=Isopropyl, X=H, Z₂=CH₃)

0.66 mL 2-bromopropane is added by drops to 220 mg magnesium filings in 1.5 mL absolute tetrahydrofuran under a nitrogen atmosphere. 15 min after the start of the Grignard reaction a solution of 500 mg narwedine, prepared in accordance with EP-A-787115, in 12 mL absolute tetrahydrofuran is added by drops under ice cooling and the mixture is stirred at room temperature. After 2 h the reaction mixtured is hydrolyzed with 30 mL water under ice cooling, acidified with 2N hydrochloric acid and stirred for 30 min at 60° C. The solution is made basic with concentrated aqueous ammonia solution and extracted three times, each time with 30 mL ethyl acetate. The combined organic phases are washed one time with a saturated aqueous sodium chloride solution, dried over sodium sulfate, filtered and concentrated. The product is purified by column chromatography (chloroform:MeOH=97:3).

Yield: 399 mg oily substance (69% of theory) R_{f}: 0.55 (chloroform:MeOH=97:3)

¹H-NMR (CDCl₃): δ 1.76-1.81 (m, 6H), 1.64 (ddd, 1H), 2.13 (ddd, 1H), 1.97 (ddd, 1H), 2.37 (ddd, 1H), 3.06 (ddd, 1H), 3.31 (ddd, 1H), 2.48 (s, 3H), 3.80 (s, 3H), 3.66 (d, 1H), 4.10 (d, 1H), 4.62 (b, 1H), 5.76 (d, 1H), 6.51 (d, 1H), 6.56 (d, 1H), 6.61 (d, 1H) ¹³C-NMR (CDCl₃): δ 19.8 (q), 20.7 (q), 26.3 (t), 34.8 (t), 48.1 (s), 53.9 (t), 41.9 (q), 55.6 (q), 60.6 (t), 128.8 (s), 89.0 (d), 122.9 (d), 110.6 (d), 121.1 (d), 121.4 (s), 124.4 (d), 130.8 (s), 133.6 (s), 143.7 (s), 146.3 (s),

In summation it can be said that the new derivatives of 4a,5,9,10,11,12-hexahydro-benzofuro[3a,3,2][2]benzazepine with the general formulas Ia, Ib and Ic not only can be produced efficiently on an industrial scale with the desired optical purity, but, because of their pharmacological activity, they are also suitable for the preparation of drugs for the treatment of many different diseases, especially diseases of the central nervous system (CNS).

1. A derivative of 4a,5,9,10,11,12-hexahydrobenzofuro[3a,3,2][2] benzazepine with the general formulas Ia or lb 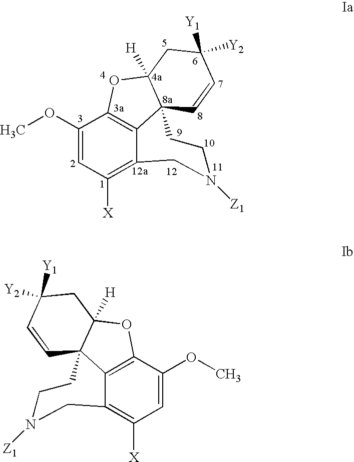
and their salts, where
Ia represents optically active (−) derivatives of galanthamine and Ib represents optically active (+) derivatives of galanthamine, which occur in a mirror configuration, and in which
Y₁ and Y₂ are alternately H or OH,
X=H or Br and
Z=a group of the following formulas 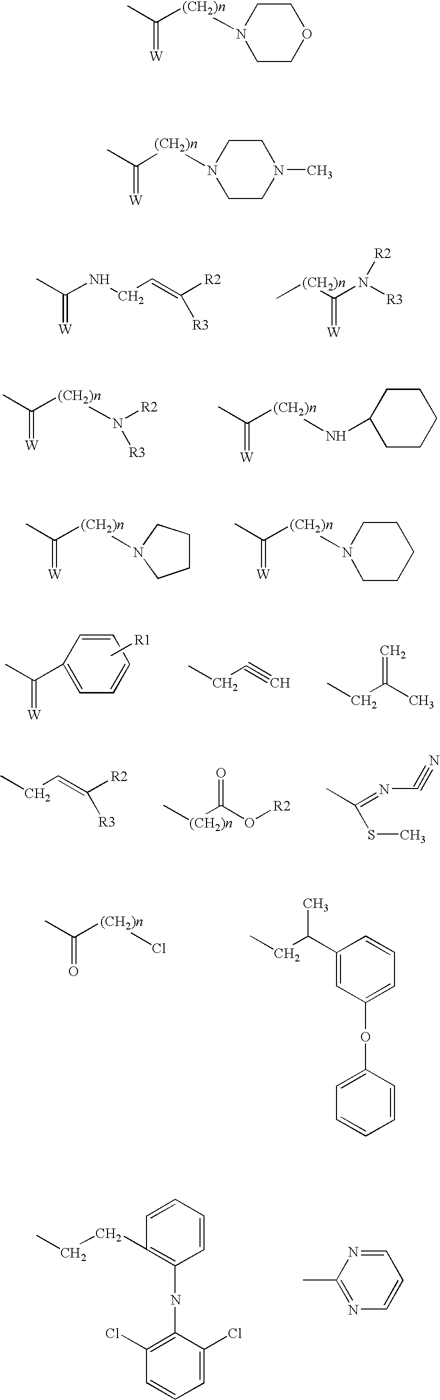 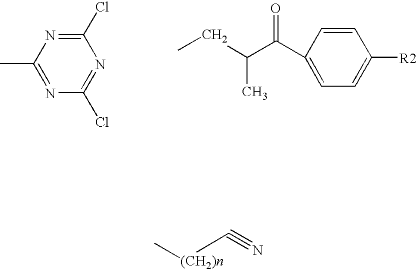
in which
R₁=H, Cl, Br, I, F, OH, linear or branched (C₁-C₆) alkyl, linear or branched (C₁-C₆) alkyloxy, NO₂, NR₂R₃,
R₂=R₃=H, linear or branched (C₁-C₆) alkyl
W=H,O, S
n=0or 1-6
in which
Z₁ is equal to H solely for compounds 1, 3, 13 and 24 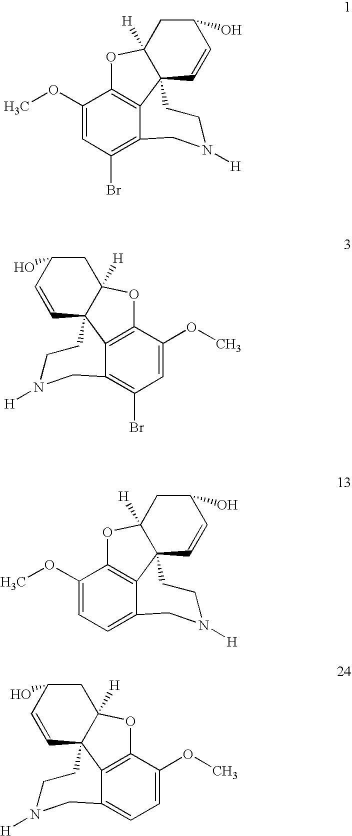
where compounds 1 and 13 are (−) derivatives of 6-epinorgalanthamine and compounds 3 and 24 are (+) derivatives of 6-epinorgalanthamine, and in which
Z₁ is equal to hydroxypropyl solely for the compound 29 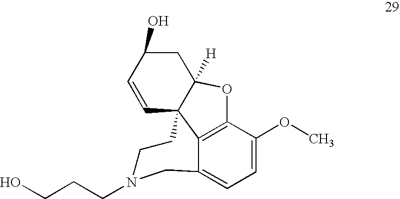
and
Z₁ is equal to ethyl solely for the compound 26 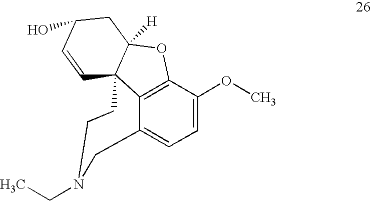
and
Z₁ is equal to methyl solely for the following compounds 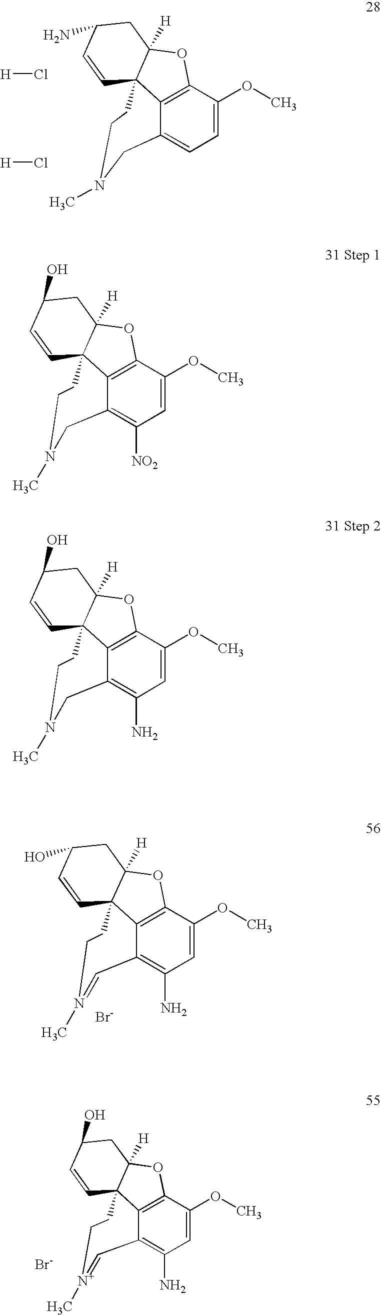
and where compounds 29, 31 and 55 are (+) derivatives of galantharnine and compounds 26, 28 and 56 are (+)-epi derivatives of galanthamine.

2. A derivative of 4a,5,9,10,11,12-hexahydrobenzofuro[3a,3,2][2]benzazepine with the general formula 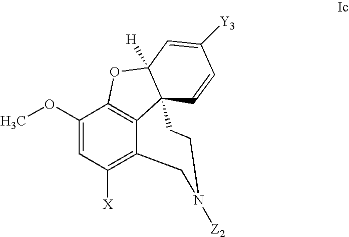
and their salts, where
X is H or Br,
Z₂ is H, linear or branched (C₁-C₆) alkyl, linear or branched (C₂-C₇) alkenyl, linear or branched (C₂-C₇) alkinyl and
Y₃ is linear or branched (C₁-C₆) alkyl, phenyl, linear or branched (C₁-C₆) alkylphenyl, nitrophenyl, chlorophenyl, bromophenyl, aminophenyl, hydroxyphenyl.

3. A method for the preparation of a compound as in claim 1 comprising treating an optically active 11-norgalanthamine derivative with dilute acid.

4. A method as in claim 3, wherein an optically active 11-norgalantharnine derivative is converted to a 6-epi derivative of galanthamine by treatment with dilute acid.

5. A method as in claim 3, wherein the steric configuration at carbon 6 is changed in the acid treatment and the steric configuration at the asymmetric carbon atoms 4a and 8a remains unaltered.

6. A method for the preparation of a compounds as in claim 1 wherein alkylation or acylation reactions are carried out in a solvent chosen from the group consisting of toluene, acetonitrile, ethanol, acetone, 2-butanone, dimethyl formamide and chloroform.

7. A method as in Claim 28, wherein the compounds with the general formula Ic are prepared from the corresponding (−)-narwedine components by alkylation in a multistep Grignard reaction.

8. A method for preparation of compounds 1, 3, 13 and 24 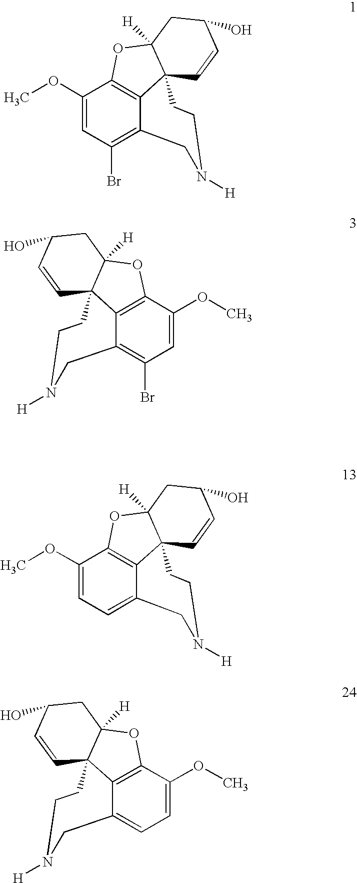
is characterized by the fact that comprising reacting the corresponding starting compounds based on norgalanthamine in the presence of a base.

9. A method as in Claim 8, wherein sodium hydrogen carbonate, potassium carbonate, sodium hydroxide, potassium hydroxide, triethylamine or pyrridine or mixtures thereof are used as base.

10. A method as in claim 8, wherein the base is used in an amount between 5 and 20 wt % with respect to 100 wt % starting product.

11. A drug comprising one or more of the compounds as in claim 1 as a pharmaceutically active agent.

12. A method for the treatment of Alzheimer's disease or related conditions of dementia comprising administering to a patient one or more compounds as in claim 1 in pure form or in the form of their pharmaceutically safe acid addition salts.

13. A method for the treatment of Parkinson's disease comprising administering to a patient one or more compounds as in claim 1 in pure form or in the form of their pharmaceutically safe acid addition salts.

14. A method for the treatment of Huntington's disease (chorea) comprising administering to a patient one or more compounds as in claim 1 in pure form or in the form of their pharmaceutically safe acid addition salts.

15. A method for the treatment of multiple sclerosis comprising administering to a patient one or more compounds as in claim 1 in pure form or in the form of their pharmaceutically safe acid addition salts.

16. A method for the treatment of amyotrophic lateral sclerosis comprising administering to a patient one or more compounds as in claim 1 in pure form or in the form of their pharmaceutically safe acid addition salts.

17. A method for the treatment of epilepsy comprising administering to a patient one or more compounds as in claim 1 in sure form or in the form of their pharmaceutically safe acid addition salts.

18. A method for the treatment of the consequences of stroke comprising administering to a oatient one or more compounds as in claim 1 in pure form or in the form of their pharmaceutically safe acid addition salts.

19. A method for the treatment of consequences of craniocerebral trauma comprising administering to a patient one or more compounds as in claim 1 in pure form or in the form of their pharmaceutically safe acid addition salts.

20. A method for the treatment and prophylaxis of the effects of diffuse oxygen and nutrient deficiency in the brain such as are observed after hypoxia, anoxia, asphyxia, cardiac arrest, intoxications, narcosis and in the infant after complications in cases of difficult birth comprising administering to a patient one or more compounds as in claim 1 in pure form or in the form of their pharmaceutically safe acid addition salts.

21. A method for the prophylactic treatment of apoptotic degeneration in neurons that have been or are being damaged by local radio- or chemotherapy of brain tumors comprising administering to a patient one or more compounds as in claim 1 in pure form or in the form of their pharmaceutically safe acid addition salts.

22. A method for the treatment of bacterial meningitis comprising administering to a patient one or more compounds as in claim 1 in pure form or in the form of their pharmaceutically safe acid addition salts.

23. A method for the treatment of diseases within an apoptotic component, comprising administering to a patient one or more compounds as in claim 1 in pure form or in the form of their pharmaceutically safe acid addition salts.

24. A method for the treatment of diabetes mellitus, comprising administering to a patient one or more compounds as in claim 1 in pure form or in the form of their pharmaceutically safe acid addition salts.

25. A method for the treatment of postoperative delirium and/or subsyndromal postoperative delirium comprising administering to a patient one or more compounds as in claim 1 in pure form or in the form of their pharmaceutically safe acid addition salts.

26. A method for the preventive treatment of postoperative delirium and/or subsyndromal postoperative delirium comprising administering to a patient one or more compounds as in claim 1 in pure form or in the form of their pharmaceutically safe acid addition salts.

27. A method as in claim 3, wherein the dilute acid is dilute hydrochloric acid.

28. A method for the preparation of a compound as in claim 2, wherein alkylation or acylation reactions are carried out in a solvent chosen from the group consisting of toluene, acetonitrile, ethanol, acetone, 2-butanone, dimethyl formamide and chloroform.

29. A drug comprising one or more of the compounds as in claim 2 as a pharmaceutically active agent.

30. A method for the treatment of Alzheimer's disease or related conditions of dementia comprising administering to a patient one or more compounds as in claim 2 in pure form or in the form of their pharmaceutically safe acid addition salts.

31. A method for the treatment of Parkinson's disease comprising administering to a patient one or more compounds as in claim 2 in pure form or in the form of their pharmaceutically safe acid addition salts.

32. A method for the treatment of Huntington's disease (chorea) comprising administering to a patient one or more compounds as in claim 2 in pure form or in the form of their pharmaceutically safe acid addition salts.

33. A method for the treatment of multiple sclerosis comprising administering to a patient one or more compounds as in claim 2 in pure form or in the form of their pharmaceutically safe acid addition salts.

34. A method for the treatment of amyotrophic lateral sclerosis comprising administering to a patient one or more compounds as in claim 2 in pure form or in the form of their pharmaceutically safe acid addition salts.

35. A method for the treatment of epilepsy comprising administering to a patient one or more compounds as in claim 2 in pure form or in the form of their pharmaceutically safe acid addition salts.

36. A method for the treatment of the consequences of stroke comprising administering to a patient one or more compounds as in claim 2 in pure form or in the form of their pharmaceutically safe acid addition salts.

37. A method for the treatment of consequences of craniocerebral trauma comprising administering to a patient one or more compounds as in claim 2 in pure form or in the form of their pharmaceutically safe acid addition salts.

38. A method for the treatment and prophylaxis of the effects of diffuse oxygen and nutrient deficiency in the brain such as are observed after hypoxia, anoxia, asphyxia, cardiac arrest, intoxications, narcosis and in the infant after complications in cases of difficult birth comprising administering to a patient one or more compounds as in claim 2 in pure form or in the form of their pharmaceutically safe acid addition salts.

39. A method for the prophylactic treatment of apoptotic degeneration in neurons that have been or are being damaged by local radio- or chemotherapy of brain tumors comprising administering to a patient one or more compounds as in claim 2 in pure form or in the form of their pharmaceutically safe acid addition salts.

40. A method for the treatment of bacterial meningitis comprising administering to a patient one or more compounds as in claim 2 in pure form or in the form of their pharmaceutically safe acid addition salts.

41. A method for the treatment of diseases within an apoptotic component comprising administering to a patient one or more compounds as in claim 2 in pure form or in the form of their pharmaceutically safe acid addition salts.

42. A method for the treatment of diabetes mellitus comprising administering to a patient one or more compounds as in claim 2 in pure form or in the form of their pharmaceutically safe acid addition salts.

43. A method for the treatment of postoperative delirium and/or subsyndromal postoperative delirium comprising administering to a patient one or more compounds as in claim 2 in pure form or in the form of their pharmaceutically safe acid addition salts.

44. A method for the preventive treatment of postoperative delirium and/or subsyndromal postoperative delirium comprising administering to a patient one or more compounds as in claim 2 in pure form or in the form of their pharmaceutically safe acid addition salts.

45. A method as in claim 23, wherein the diseases with an apoptic component are accompanied by amyloid-associated cell degeneration.

46. A method as in claim 41, wherein the diseases with an apoptic component are accompanied by amyloid-associated cell degeneration.

47. A method as in claim 24, wherein the diabetes mellitus is accompanied by amyloid degeneration of the islet cells.

48. A method as in claim 42, wherein the diabetes mellitus is accompanied by amyloid degeneration of the islet cells.
